(19) United States
(12) Patent Application Publication
(10) Pub. No.: US 2022/0330861 A1
(43) Pub. Date: Oct. 20, 2022

(54) Self-Contained Dermal Patch for Detection of Physiological Analytes

(71) Applicant: Satio, Inc., Weston, MA (US)

(72) Inventor: Namal NAWANA, Weston, MA (US)

(73) Assignee: Satio, Inc., Weston, MA (US)

(21) Appl. No.: 17/719,881

(22) Filed: Apr. 13, 2022

Related U.S. Application Data
(60) Provisional application No. 63/174,956, filed on Apr. 14, 2021.

(51) Int. Cl. A61B 5/15; A61B 5/00; A61B 5/145

(52) U.S. Cl. CPC A61B 5/150969; A61B 5/6833; A61B 5/0022; A61B 5/7435; A61B 5/746; A61B 5/150984; A61B 5/14546

(57) ABSTRACT

In one aspect, a dermal patch is disclosed, which comprises at least a pair of sensing units each configured for detecting at least one analyte in a physiological sample, at least one microneedle configured for puncturing the skin to allow collection of the physiological sample, and a selector device for selecting any one of said sensing units for receiving at least a portion of said collected physiological sample for analysis thereof.

### CROSS REFERENCE TO RELATED APPLICATIONS

This application claims priority to and the benefit of U.S. Provisional Application 63/174,956 filed on Apr. 14, 2021, the entire teachings of this provisional application is incorporated by reference herein.

### BACKGROUND

The present teachings are generally directed to dermal patches that can be employed to collect a physiological sample from a subject and optionally analyze the sample so as to detect and optionally quantify a variety of target analytes, such as a variety of biomarkers.

Biomarkers are increasingly employed for diagnosis of various disease conditions as well as for assessing treatment protocols. In many cases, it is important to monitor the level of a biomarker over time to assess, e.g., the progression of a disease condition. Such temporal monitoring of biomarkers using conventional techniques for sample collection can be cumbersome and painful for the patient. For example, the invasive nature of drawing a blood sample from a patient can cause discomfort and may lead to less cooperation from a subject, especially children, and hence render multiple measurements of a target analyte difficult.

Some recently-developed applicator devices that allow continuous monitoring of certain analytes, such as glucose, fail to provide a solution for improving the detection and measurement of the levels of a target analyte at a plurality of discrete times in small volumes of a physiological sample (e.g., blood) extracted from a subject. Further, such conventional applicators typically suffer from a number of shortcomings, such as, low sensitivity and/or specificity.

### SUMMARY

In one aspect, a dermal patch is disclosed, which includes at least a pair of sensing units each configured for detecting at least one target analyte in a physiological sample, and at least one microneedle that is configured for puncturing the skin to allow collection of the physiological sample. The dermal patch can further include a selector device for selecting any one of said sensing units for receiving at least a portion of the collected physiological sample for analysis thereof.

In some embodiments, the selector device may be implemented as any of a mechanical, electromechanical, or electromagnetic selection element.

In some embodiments, the selector device includes at least one visual indicator, e.g., a selector dial, for allowing a user to select any one of said sensing units, e.g., at different discrete times.

The dermal patch can further include a plurality of reservoirs for storing one or more reagents/buffers for processing the physiological sample to generate a processed sample, where each of said reservoirs is associated with one of said sensing units. By way of example, the processing reagents/buffers may be selected for processing of a blood sample (e.g., the reagents may include an anticoagulant, such as heparin, and/or a protease inhibitor). In some embodiments, the processing reagents (e.g., primers, etc.) are suitable for providing isothermal amplification of a target analyte (e.g., a cell free DNA segment in the blood sample). In some embodiments, the processing reagents can be in a lyophilized form when stored in the reservoir and can be reconstituted when transferred to a sample collection chamber, or before transfer. For example, the sample collection chamber may include a solvent for reconstituting the lyophilized reagents. Alternatively, such a solvent may be stored in a pouch and can be released into a reservoir in response to the selection of a sensing unit associated with that reservoir. In some embodiments, the lyophilized reagent(s) may be stored on a nitrocellulose pad.

In many embodiments, the reservoirs are pre-filled with the requisite processing reagents/buffers such that the dermal patch can be used without a need to fill the reservoirs with the processing reagents/buffers at the point of care. In other words, a user can utilize the dermal patch with all the requisite buffers and processing reagents on board. This feature provides distinct advantages in that it ensures consumer safety and reduces, and preferably eliminates, the risk of error. In other words, in many embodiments a dermal patch according to the present teachings contains all the necessary sample processing reagents/buffers for its intended use. Further, in many embodiments, the dermal patch can include the electronic circuitry that allows the processing of signals generated by one or more sensors of the dermal patch.

As discussed further below, in some embodiments, of a dermal patch according to the present teachings, the processing reagent(s) and/or buffers required for detecting and optionally quantifying a target analyte may be stored in the sample collection chambers. For example, the processing reagent(s) may be stored in the sample collection chambers in a lyophilized form (e.g., on a nitrocellulose pad), or otherwise. In some such embodiments, the dermal patch does not include any reservoirs, or at least some of the sample collection chambers are not associated with a reservoir, and all the requisite processing reagent(s) and/or buffers, etc. are stored in the sample collection chambers. Alternatively, in some embodiments, all the requisite processing reagent(s)/buffers are stored in a lyophilized form in the sample collection chambers and one or more solvents required for reconstitution of the lyophilized reagent(s) may be stored in one or more reservoirs incorporated in the dermal patch (such as those disclosed herein), where in use the solvent(s) may be transferred from the reservoir(s) to the sample collection chambers. e.g., in a manner disclosed herein, for the reconstitution of the lyophilized reagent(s) stored therein.

In some embodiments, the site for application of the dermal patch is envisaged to be the forearm, or the upper arm. In some implementations, such application of the dermal patch can advantageously reduce or eliminate the need for electronics on board. In some embodiments, a wearable unit, e.g., a watch-like device, may be employed to supply power to one or more sensors of the dermal patch and/or receive signals generated by those sensor(s) for analysis and/or presentation of the analysis results to a user (the subject and/or a healthcare professional). Further, in some embodiments, the wearable device may include communication circuitry for communicating the data and/or analysis results to another device, such as an external server. By way of example, the wearable device may employ a wireless communication protocol, such as Bluetooth, Wi-Fi, etc., for communicating with the external device.

A variety of sensors may be incorporated in a dermal patch according to the present teachings. The sensors may be passive or active sensors. Some examples of sensors include, without limitation, sensors that provide chromatographic or “photo-visual,” or digital readouts. For example, such a sensor may be a colorimetric sensor, e.g., an immunoassay sensor including lateral flow sensors, as well as isothermal amplification detection systems. Some examples of other suitable sensors include, without limitation, graphene-based sensors, electrochemical sensors, and chemical sensors, among others.

Each of the sensing units may include a sample collection chamber for receiving at least a portion of said physiological sample in response to selection of the sensing unit by said selector device. In some embodiments, a sample collection chamber of a dermal patch according to the present teachings may have a volume equal to or less than about 2 milliliters, or equal to or less than about 1 milliliter, e.g., in a range of about 10 microliters to about 1 milliliter or about 100 microliters to about 500 microliters to about 800 microliters.

The dermal patch can further include at least one fluid channel having an inlet configured to receive the physiological sample through the punctured skin and an outlet through which the received sample can be introduced into a sample collection chamber of a selected one of said sensing units.

In some embodiments, the dermal patch can include a switch for selectively establishing a fluid path between the outlet of the above fluid channel and a selected one of the sample collection chambers. A controller can be in communication with the switch for activating the switch in accordance with a predefined temporal schedule to collect multiple physiological samples at different times.

In some embodiments, the switch may include a plurality of internal channels, where in one position of the switch one of the internal channels directs the received physiological sample into one of the sample collection chambers and in another position of the switch another one of the internal channels directs the received physiological sample into another one of the sample collection chambers. A variety of switches, such as mechanical, electromechanical and electromagnetic switches, may be employed.

In some embodiments, a fluid transfer channel is coupled to the selector device for establishing a fluid path between a selected sensing unit and a reservoir associated therewith. In some embodiments, the transfer of a processing fluid stored in a reservoir to the respective sample collection chamber may be achieved passively or actively. By way of example, in some embodiments, each of the reservoirs and a respective sample collection chamber are positioned relative to one another such that gravity can facilitate the transfer of the processing fluid stored in that reservoir to the sample collection chamber.

In some embodiments, one or more magnetic beads may be stored in at least one of the sample collection chambers, where the magnetic beads may be activated via an external magnet to cause mixing of the physiological sample and the processing fluid introduced into said at least one sample collection chamber.

Each of the sensing units can include at least one sensor that is in fluid communication with the sample collection chamber of that sensing unit, or can be brought into fluid communication with that sensing unit, for coming into contact with at least a portion of the processed sample and to generate one or more signals in response to the detection of a target analyte, when present in the sample. By way of example, the sensor can be coupled to the sample collection chamber via a sealed opening. Alternatively, the sample collection chamber may be formed of a flexible material that expands upon receiving the sample and the processing fluid so as to open a slit, thereby providing a fluid path between the sensor and the sample collection chamber. Other suitable means for interrogating a sample may also be employed. By way of example, in some cases, the interrogation of a sample may be achieved without the need for direct contact between a sensor and the sample, e.g., via optical techniques, such as fluorescent and/or Raman techniques.

In some embodiments, the dermal patch may include a computer system that is in communication with the sensors of the dermal patch for receiving one or more signals (e.g., detection signals) generated by the sensors. The circuitry may be configured to process the signals to determine the presence of a target analyte in the sample and optionally quantify the level of the target analyte, when present in the sample.

A variety of sensors can be incorporated in a dermal patch according to the present teachings. Some examples of such sensors include, without limitation, graphene-based sensors, electrochemical sensors, colorimetric sensors, and/or optical sensors. In some embodiments, a colorimetric sensor may employ an immunoassay for the detection of the target analyte. In some embodiments in which a colorimetric sensor is employed, at least a portion of the dermal patch may include a transparent portion to allow the visualization of the sensor.

The circuitry may be implemented using the techniques known in the art as informed by the present teachings. By way of example, the circuitry may include at least one memory module for storing the signals generated by the sensors of the dermal patch. The circuitry may be configured to process the stored signals, e.g., detection signals, generated by different sensing units to determine a variation, if any, of a target analyte level at a plurality of discrete time at which the sensing units are activated. The circuitry may also include a communication module to allow communication between the circuitry and an external electronic device. Such an external electronic device may be a mobile electronic device. By way of example, in some embodiments, a variety of wireless communication protocols may be used for transmitting data from the circuitry to the external electronic device. Some examples of such wireless communication protocols may include Bluetooth, Wi-Fi, and BTLE protocol for establishing a communication link between said patch and said electronic device.

In some embodiments, the physiological sample may include any of blood and/or interstitial fluid.

In some embodiments, the target analyte may be a biomarker, e.g., a biomarker that may be indicative of a disease condition, e.g., organ damage. In some embodiments, the biomarker may be indicative of a traumatic brain injury, including a mild traumatic brain injury. Some example of such a biomarker include, without limitation, any of myelin basic protein (MBP), ubiquitin carboxyl-terminal hydrolase isoenzyme L1 (UCHL-1), neuron-specific enolase (NSE), glial fibrillary acidic protein (GFAP), and S100-B.

In other embodiments, the dermal patch may be configured for the detection of other biomarkers, such as troponin, BNP, and HbA1C, among others.

In some embodiments, the sensing units of a dermal patch according to the present teachings may be configured to detect the same target analyte while in other embodiments the sensing units may be configured to detect different target analytes.

The number of sensing units can vary based on a particular application. By way of example, and without limitation, the number of sensing units may be in a range of 2 to about 20, e.g., 6 to 10, though other numbers may also be used.

In some embodiments, the dermal patch may include a plurality of microneedles, e.g., in a range of 10 to 20. In some embodiments, the microneedles have a length in a range of about 100 microns to about 1500 microns in length, and have a width of about 50 to about 250 microns, and about 1-25 microns in diameter, though other sizes may also be employed.

In some embodiments, the microneedle(s) may be movable between a retracted position and a deployed position in which the microneedle(s) are capable of puncturing the skin. The dermal patch may also include an actuation mechanism operably coupled to the microneedles for transitioning the microneedles between the retracted position and the deployed position.

In some embodiments, the dermal patch may include one or more pumps that are coupled to one or more internal fluidic channels of the dermal patch for generating a negative pressure therein in order to facilitate the flow of a sample and/or a processing fluid therethrough. In some such embodiments, the pump(s) can be positive displacement pumps.

The dermal patch may further include an adhesive layer that facilitates the attachment of the dermal patch to a patient's skin.

In a related aspect, a dermal patch is disclosed, which includes at least two sample collection chambers, each configured for receiving a physiological sample collected from a subject. The dermal patch further includes at least one reservoir for storing one or more processing reagents/buffers for processing the physiological sample so as to provide a processed sample. The dermal patch can also include at least two detection units each operably coupled to one of said sample collection chambers for detecting a target analyte, when present in the collected sample. The detection units can include one or more sensors, such as those discussed herein. The sensors may be configured to provide a quantitative level of a target analyte, when present in the sample. Alternatively, an external circuitry may be utilized to quantify the level of the target analyte based on signals generated by the sensor.

Further, the dermal patch may include a selector device for selecting any of said sample collection chambers for receiving the physiological sample. One or more microneedles may be incorporated in the dermal patch for puncturing a subject's skin to allow collecting a physiological sample.

Further understanding of various aspects of the present teachings may be obtained by reference to the following detailed description

### BRIEF DESCRIPTION OF THE DRAWINGS

FIG. 1 is a schematic view of an embodiment of a dermal patch according to the present teachings,

FIG. 2 is an exploded view of the dermal patch depicted in FIG. 1,

FIG. 3 is a schematic view of the dermal patch of FIG. 1 illustrating, among other elements, a plurality of internal channels and sample collection chambers,

FIG. 4 is a schematic front view of the dermal patch of FIG. 1,

FIG. 5 schematically depicts an implementation of a controller for use in some embodiments of a dermal patch according to the present teachings,

FIG. 6 is a schematic view of another embodiment of a dermal patch according to the present teachings in which a plurality of pumps, such as positive displacement pumps, are employed for facilitating the flow of a collected sample through various internal channels of the dermal patch,

FIG. 7A is a schematic view of another embodiment of a dermal patch according to the present teachings in which a programmable switch is incorporated for directing a sample drawn from a subject to sample collection chambers of the dermal patch in accordance with a predefined temporal schedule,

FIG. 7B schematically depicts an example of an implementation of the switch depicted in FIG. 7A,

FIG. 8 schematically depicts an example of an actuation mechanism for moving the microneedles of a dermal patch according to an embodiment of the present teachings between a retracted position and a deployed position,

FIG. 9 schematically depicts a dermal patch that includes a selector device having a visual indicator for allowing a user to select one of a plurality of sensing units for receiving a physiological sample,

FIG. 10 shows four sample collection chambers that may be incorporated into a dermal patch according to some embodiments of the present teachings,

FIG. 11 is a partial schematic view of a dermal patch according to an embodiment in which two sample collection chambers share a reservoir in which one or more sample processing reagents/buffers are stored,

FIG. 12 schematically shows that in some embodiments a dermal patch may be attached to an arm of a subject and be powered by a wearable device and/or transmit data thereto,

FIG. 13 shows schematically an inductive magnetic coupling between two coils one of which is incorporated in a dermal patch according to an embodiment and another one is incorporated in the wearable device;

FIG. 14 schematically depicts a smart phone that is in communication with an electronic medical record (“EMR”) database and a dermal patch with a quick response (“QR” code) in accordance with an exemplary embodiment;

FIG. 15 schematically depicts a cloud computing environment in accordance with an exemplary embodiment;

FIG. 16 depicts a method for updating an EMR in accordance with an exemplary embodiment;

FIG. 17 schematically depicts a dermal patch with a locking mechanism, an actuation button, and an electromechanical actuator in accordance with an exemplary embodiment;

FIG. 18 schematically depicts a dermal patch with a skin sensor in accordance with an exemplary embodiment;

FIG. 19 depicts a method for deploying microneedles of a dermal patch in accordance with an exemplary embodiment;

FIG. 20 depicts a method for unlocking a dermal patch in accordance with an exemplary embodiment;

FIG. 21 depicts another method for deploying microneedles of a dermal patch in accordance with an exemplary embodiment;

FIG. 22 depicts another method for unlocking a dermal patch in accordance with an exemplary embodiment;

FIG. 23 depicts two smart phones including a medical professional's smart phone connected to a dermal patch in accordance with an exemplary embodiment;

FIG. 24 depicts another method for deploying microneedles of a dermal patch in accordance with an exemplary embodiment;

FIG. 25 depicts another method for unlocking a dermal patch in accordance with an exemplary embodiment;

FIG. 26 depicts a metaverse network in accordance with an exemplary embodiment;

FIG. 27 depicts a computer system with a metaverse client in accordance with an exemplary embodiment; and

FIG. 28 depicts a metaverse in accordance with an exemplary embodiment.

### DETAILED DESCRIPTION

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

The present teachings are generally directed to dermal patches that may be utilized, for example, to measure the level of one or more target analytes in a physiological sample, e.g., a blood sample and/or an interstitial fluid. In some embodiments, a dermal patch according to the present teachings allows the collection and analysis of a plurality of physiological samples at different times, thereby facilitating the diagnosis and/or prognosis of a disease condition and/or efficacy of a therapeutic treatment. In many embodiments, a dermal patch according to the present teachings includes the requisite processing reagents/buffers (herein referred to as “processing fluid”) for processing a received sample on board. Such a feature, that is, the incorporation of the required processing reagents in the dermal patch, can provide a number of advantages, including additional safety as well as enhanced sensitivity and/or specificity. Further, in some embodiments, a dermal patch according to the present teachings allows measuring the level of a target analyte at a plurality of discrete times, thereby providing valuable information regarding the occurrence, progression, and/or amelioration of a disease condition.

Various terms are used herein in accordance with their ordinary meanings in the art, unless indicated otherwise. The term “about,” as used herein, denotes a deviation of at most 10% relative to a numerical value. The term “substantially,” as used herein, refers to a deviation, if any, of at most 10% from a complete state and/or condition. The terms “needle” and “microneedle” are used herein to broadly refer to an element that can provide a passageway, or facilitate the production of a passageway, for collecting a physiological sample, such as a blood or an interstitial fluid sample through a patient's skin, e.g., via puncturing the subject's skin.

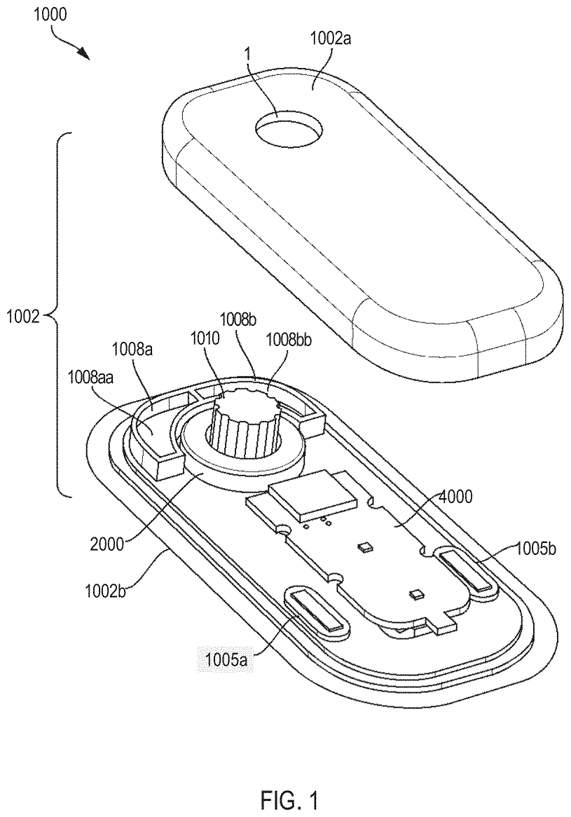

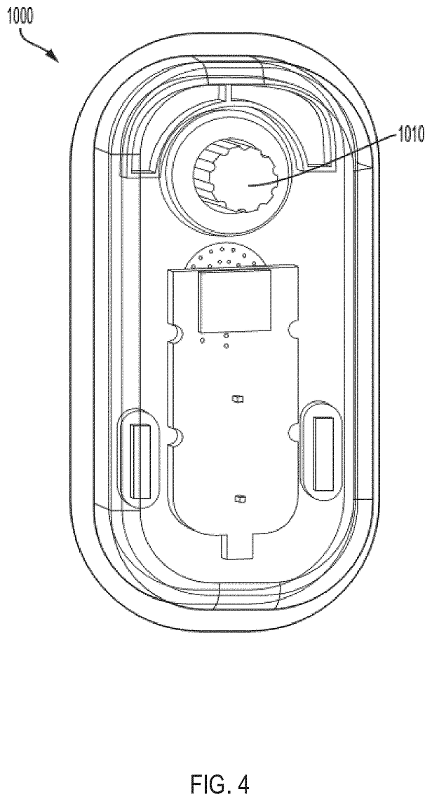

With reference to FIGS. 1, 2, 3, and 4, a dermal patch 1000 according to an embodiment of the present teachings includes a housing 1002 having a top portion 1002a and a bottom portion 1002b, which can be coupled to one another, e.g., releasably or otherwise (e.g., via glue, fasteners, etc.). While in this embodiment the housing 1002 is formed of two portions that are coupled to one another, in other embodiments the housing 1002 may be formed as a single integral unit.

The housing 1002 may be formed of any suitable polymeric material. By way of example, and without limitation, the housing 1002 may be molded from polymeric materials, such as, but not limited to, polyolefins, PET (Polyethylene Terephthalate), polyurethanes, polynorbornenes, polyethers, polyacrylates, polyamides (Polyether block amide also referred to as Pebax®), polysiloxanes, polyether amides, polyether esters, trans-polyisoprenes, polymethyl methacrylates (PMMA), cross-linked trans-polyoctylenes, cross-linked polyethylenes, cross-linked polyisoprenes, cross-linked polycyclooctenes, inorganic-organic hybrid polymers, co-polymer blends with polyethylene and Kraton®, styrene-butadiene co-polymers, urethane-butadiene co-polymers, polycaprolactone or oligo caprolactone co-polymers, polylactic acid (PLLA) or polylactide (PL/DLA) co-polymers, PLLA-polyglycolic acid (PGA) co-polymers, and photocrosslinkable polymers.

In some embodiments, the housing 1002 or at least a portion thereof may be formed of a transparent polymeric material, e.g. PDMS, to allow visibility of at least a portion of components disposed within the housing. By way of example, as discussed in more detail below, in some embodiments in which a colorimetric sensor is employed the transparent portion can allow visualization of a color generated by the colorimetric sensor.

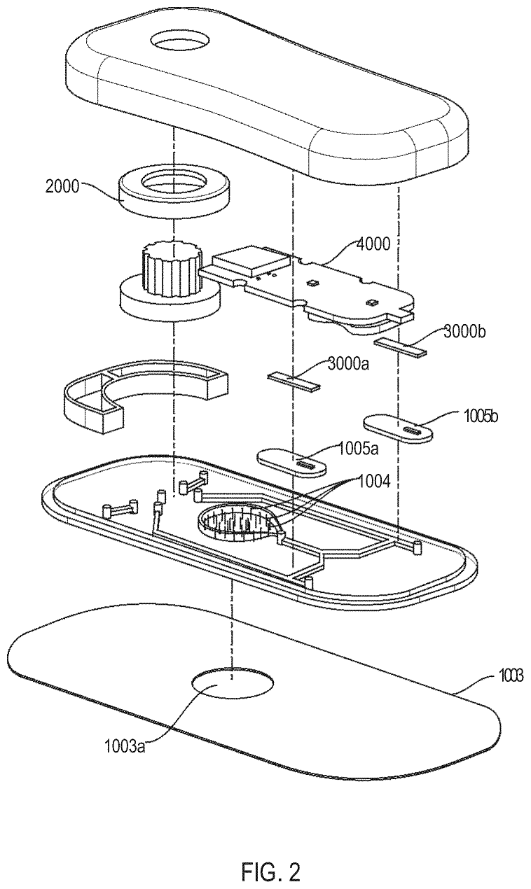

The dermal patch 1000 includes an adhesive layer 1003 that allows attaching the dermal patch to a subject's epidermal layer (See, e.g., FIG. 2).

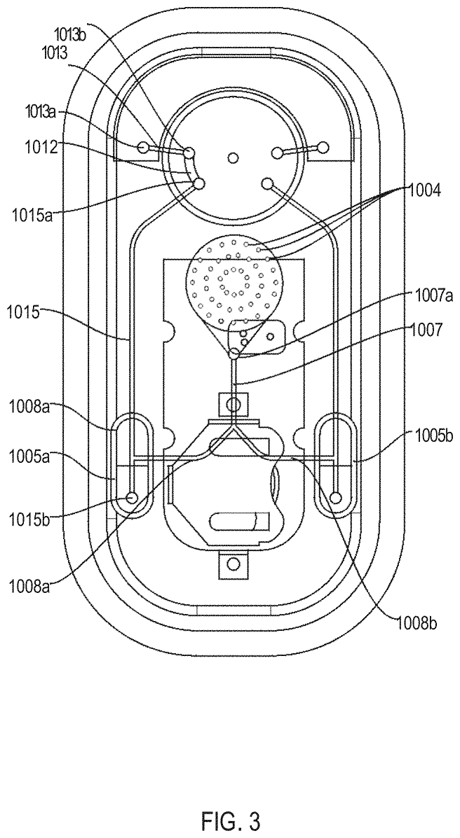

With particular reference to FIGS. 2 and 3, in this embodiment the dermal patch 1000 includes a plurality of needles 1004 (herein also referred to as microneedles) that are in register with an opening 1003a provided in the adhesive layer 1003 to allow their contact with a subject's skin upon attachment of the dermal patch to a patient's skin.

The microneedles are configured to puncture a subject's skin and penetrate through a subject's stratum corneum and at least a portion of the epidermal layer to allow collecting a physiological fluid, e.g., capillary blood and/or inerstitial fluid. As discussed in more detail below, in some embodiments, the microneedles 1004 may be movable between a retracted position in which the microneedles are moved into a chamber within the dermal patch and a deployed position (herein also referred to as an extended position) in which the microneedles are exposed for puncturing the skin. In some embodiments, the microneedles 1004 may be formed of a polymeric material with a channel, e.g., a central channel, that allows collecting a physiological sample, e.g., a capillary blood sample and/or interstitial fluid. Some examples of suitable polymers include, without limitation, PDMS, epoxy siloxane polymer, among others. By way of example, in some embodiments, the needles can include an epoxy siloxane polymer layer that is sandwiched between two PDMS layers. In some cases, the polymeric needles can be fabricated using a mold.

With particular reference to FIGS. 2 and 3, in this embodiment the dermal patch 1000 includes two sample collection chambers 1005a and 1005b, each of which can receive a portion of a physiological sample drawn through the punctured skin. In particular, a fluidic channel 1007 having an inlet 1007a positioned behind the microchannels can receive the physiological sample drawn through the skin. The fluidic channel 1007 leads to two fluidic branches 1008a and 1008b, each of which is in fluidic communication with one of the sample collection chambers 1005a and 1005b to split a received physiological sample between the two sample collection chambers.

In some embodiments, the collection of the physiological sample through the punctured skin occurs passively while in other embodiments a negative pressure may be generated in the fluidic channel 1007 to facilitate the extraction of the physiological sample through the punctured skin and its transfer to the sample collection chambers.

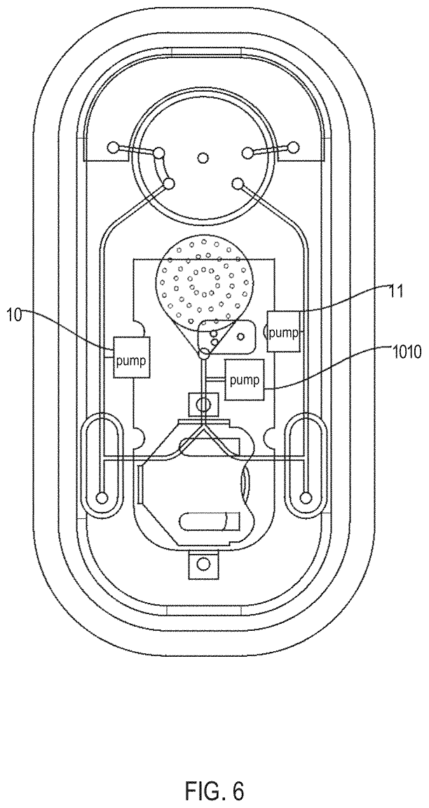

By way of example, a pump 1010 (See, e.g., FIG. 6), such as a mechanical or an electromechanical pump, may be incorporated in the dermal patch in communication with the fluidic channel 1007 to apply a negative pressure to the fluidic channel, thereby facilitating the extraction of the physiological sample through the punctured skin into the fluidic channel 1007. By way of example and without limitation, the electromechanical pump may be, e.g., a positive displacement pump.

Referring to FIGS. 1 and 2, in this embodiment, the dermal patch 1000 further includes two reservoirs 1008a and 1008b for storing one or more processing reagents/buffers (herein also referred to as a processing fluid) 1008aa/1008bb for processing the physiological samples collected within the sample collection chambers 1005a and 1005b. In this embodiment, each of the reservoirs 1008a and 1008b is associated with one of the sample collection chambers.

A variety of processing reagents/buffers known in the art may be employed in the practice of the present teachings. By way of example, the processing fluid may be selected to facilitate the detection of a biomarker of interest. By way of example, in some embodiments, the processing fluid for processing a drawn blood sample may include an anticoagulant, such as heparin. In addition, in some cases the processing fluid may include a protease inhibitor. In yet other embodiments, the processing fluid may include reagents (such as primers, etc.) that allow isothermal amplification, e.g., for detecting cell free circulating DNA. A variety of reagents and techniques for processing of a physiological samples, such as a blood sample or an interstitial fluid, known in the art may employed in the practice of the present teachings.

With particular reference to FIGS. 1 and 2, a selector device 1010 allows selectively connecting each of the reservoirs to its associated sample collection chamber. In this embodiment, the selector device 1010 is in the form of a selector dial that protrudes through an opening 1 provided on the top portion of the dermal patch's housing. A retaining ring 2000 facilitates the coupling of the selector device to the housing.

With particular reference to FIG. 3, in this embodiment, the selector device 1010 includes a fluidic transfer channel 1012 (e.g., a tube), which can be positioned, via rotation of the selector dial, into two positions, where in one position the fluidic transfer channel 1012 helps establish a fluidic path between one of the reservoirs 1008a/1008b and its respective sample collection chamber, and in the other position the fluidic transfer channel 1012 helps establish a fluidic path between the other reservoir and its respective sample collection chamber.

By way of example, FIG. 3 shows a position of the selector device in which the fluidic transfer channel 1012 provides a fluidic bridge between an output port 1013b of a fluidic channel 1013, which is configured to receive the processing fluid stored in the reservoir 1008a via its inlet port 1013a, and an inlet port 1015a of a fluidic channel 1015, which extends to an outlet port 1015b that is fluidically coupled to the sample collection chamber 1008a to help establish a fluidic path between the reservoir 1008a and the sample collection chamber 1005a.

In this manner, the processing fluid stored in the reservoir 1008a can be transferred to the respective sample collection chamber 1005a. In this embodiment, such transfer of the processing fluid from the reservoir 1008a to the respective sample collection chamber 1005a is facilitated by gravity. For example, a subject wearing the patch on her arm may be placed in a seated position with the arm extending downwardly so as to allow the force of gravity to help transfer at least a portion of the processing fluid stored in the chamber 1008a to the respective sample collection chamber 1005a. Alternatively, one or more pumps, such as pumps 10 and 11 shown schematically in FIG. 6 (e.g., positive displacement pumps) may be incorporated in the dermal patch to facilitate the transfer of the processing fluid from each reservoir to a respective sample collection chamber.

With particular reference to FIG. 2, in this embodiment, the dermal patch further includes two sensing units 3000a and 3000b (herein collectively referred to as the sensing units 3000) which are coupled, respectively, to the sample collection chambers 1005a and 1005b, and are configured for the detection of a target analyte in the received physiological samples. While in this embodiment the sensing units 3000 are shown as being coupled to the sample collection chambers in a horizontal direction (i.e., along a direction parallel to the outer surfaces of the top and bottom portions of the housing), in other embodiments, the sensing units 3000 may be positioned in an orthogonal (or any other suitable orientation).

In some embodiments, the volume of each of the sample collection chambers may be equal to or less than about 2 milliliters, or equal to or less than about 1 milliliter, or equal to or less than about 0.5 milliliters, e.g., in a range of about 10 microliters and about 1 milliliter or in a range of about 100 microliters to about 500 microliters. In many embodiments, the volume of a physiological sample, (e.g., a blood sample) drawn from a subject may be less than about 1 milliliter.

In this embodiment, each of the sensing units is in fluid communication with the respective sample collection chamber, e.g., via a sealed opening. In this embodiment, each of the sensing units includes a single sensor. In some implementations, the sensor associated with the two sample collection chambers are configured to detect the same analyte while in other implementations, the sensor associated with one sample collection chamber is configured to detect one target analyte, and the sensor associated with the other sample collection chamber is configured to detect a different analyte.

Further, in some embodiments, at least one of the sensing units may include a plurality of sensors. In some such embodiments, the plurality of sensors may be configured to detect the same target analyte, while in other embodiments the plurality of sensors may be configured to detect two or more different target analytes.

A variety of sensors may be employed in the practice of the present teachings. Some examples of suitable sensors include, without limitation, graphene-based sensors, electrochemical sensors, colorimetric sensors (e.g., sensors that employ immunoassays for the detection of a target analyte), optical sensors, among others.

In some embodiments, the sensors are configured to provide a signal indicative of the presence of a target analyte at a concentration level above a limit-of-detection (LOD) of that sensor for that target analyte. In other embodiments, the sensor may be calibrated to provide a quantitative level of the target analyte (e.g., the concentration of the target analyte in the collected sample). In addition or instead, the signals generated by a sensor may be processed via an on-board processor (as discussed further below) or an external processor to quantify the level of the target analyte detected in the sample. By way of example, such quantification may be implemented using previously-generated calibration data in a manner known in the art as informed by the present teachings.

By way of example, a target analyte (e.g., a biomarker such as those disclosed herein) may be detected via a graphene-based sensor that includes a graphene layer that is functionalized with a moiety (e.g., an antibody, an aptamer, an oligonucleotide, etc.) that exhibits specific binding to that target analyte (e.g., a protein, a DNA segment) such that upon binding of the target analyte to that moiety an electrical property of the underlying graphene layer changes, thus indicating the presence of the target analyte in the sample. Some examples of suitable graphene-based sensors are disclosed in U.S. Pat. Nos. 10,782,285, 10,401,352, 9,664,674, as well as published U.S. Patent Applications Nos. 20200011860, and 20210102937, each of which is herein incorporated by reference in their entirety.

By way of example, the detection of a target analyte may be achieved by using a graphene-based sensor and/or an electrochemical sensor that is functionalized with a probe, such as an antibody and/or an aptamer, which exhibits specific binding to that target analyte, though other sensing technologies may also be utilized.

In another embodiment, the sensor can be an electrochemical sensor that can function in a faradaic or non-faradaic mode to detect a target analyte of interest. For example, such an electrochemical sensor may include a working electrode, a reference electrode and a counter electrode. By way of example, in some embodiments, the reference electrode may be functionalized with a moiety that exhibits specific binding to a target analyte such that upon binding of that target analyte, when present in the sample, to the moiety, a change in the current through the circuit may be detected.

Other types of sensors may be employed. For example, in some embodiments, an immunosensor that employs antibodies and provide visual indication of the presence of a target analyte in a sample via a change in color may be employed. Yet, in other embodiments, optical sensors, such as fluorescent and Raman detectors, may be used.

With particular reference to FIGS. 1 and 2, the dermal patch 1000 includes a circuitry 4000, implemented on a printed circuit board (PCB), that is in communication with the sensors 3000a and 3000b such that the circuitry 4000 receives the signals generated by the sensors 3000. The connection between the circuitry 4000 and the sensors 3000 may be established via any of a wired or wireless protocol. In some implementations, the circuitry and/or the sensors 3000 can be supplied with power via an on-board power supply, e.g., a battery, incorporated, e.g., on the circuitry. Alternatively, in some implementations, the circuitry and/or the sensors can be provided with power via an external device, e.g., a wearable device. Such transfer of power from an external device may be achieved using techniques known in the art, such as inductive coupling between two elements (e.g., two coils) provided in the dermal patch and the external device.

The circuitry 4000 may be configured to process the signals generated by the sensors 3000 to determine the presence and optionally quantify the level of a target analyte in the sample. The circuitry may be implemented according to known techniques in the art as informed by the present teachings. For example, the circuitry may include an ASIC that is configured for processing the signal data generated by the sensors. The circuitry can further include one or more memory modules for storing, for example, instructions for processing the data generated by the sensors. In some embodiments, the circuitry 4000 may transmit data (e.g., data related to the level (e.g., concentration) of a target analyte in a physiological sample) to an external device, such as a mobile phone, a server, for example, for presentation to a user (e.g., a patient and/or a healthcare professional), for further analysis and/or storage. In some embodiments, the circuitry is configured to communicate the data via a wireless protocol, such as Bluetooth, Wi-Fi, and BTLE protocol, though any other suitable protocol may also be employed.

In the above embodiments, the physiological sample is drawn into the sample collection chambers, and the selector device 1010 is used for selective fluidic coupling of one of the reservoirs to a respective sample collection chamber. In other words, in the above embodiment, while both sample collection chambers receive portions of the sample substantially concurrently, the interrogation of the sample portions received in the two sample collection chambers can be done at different times.

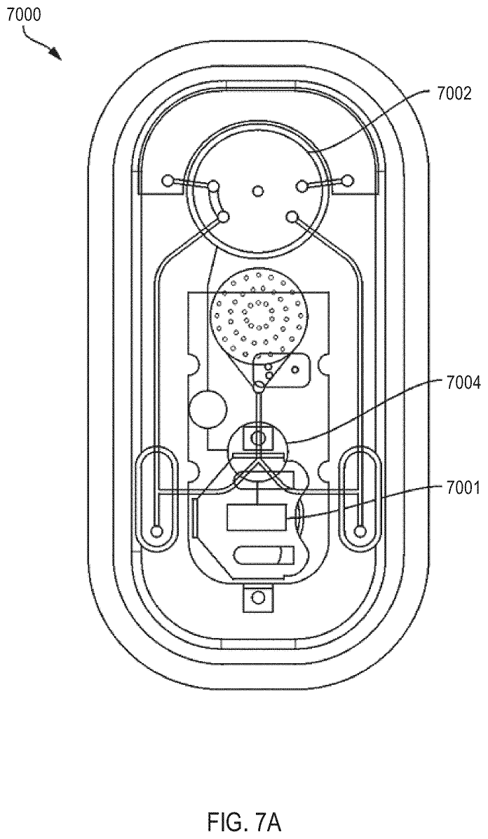

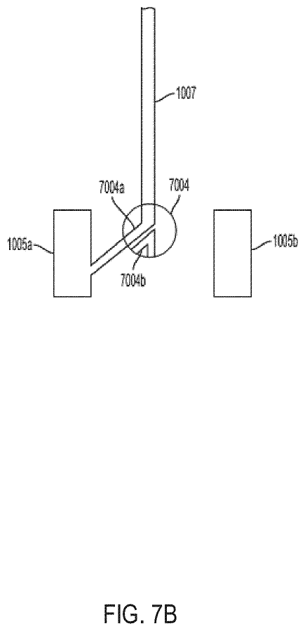

In other embodiments, the dermal patch may be configured such that different samples can be collected into the two sample collection chambers at different times. By way of example, the selector device can activate a switch to direct a physiological sample drawn through the punctured skin into one or the other of the two sample collection chambers. By way of example, FIG. 7A schematically depicts a dermal patch 7000 according to such an embodiment in which a selector device 7002 is configured to activate a switch 7004 in response to the selection of one of two sample collection chambers 7006a/7006b, e.g., via a relay 7005, to direct a physiological sample drawn through a subject's punctured skin into the selected sample collection chamber.

By way of example, with reference to FIG. 7B, in this embodiment, the switch 7004 includes internal channels 7004a/7004b, which can be selectively coupled to the outlet of the fluidic channel 1007, e.g., via rotation of a platform on which the fluidic channels are disposed, so as to direct a physiological sample flowing through the fluidic channel 1007 into the sample collection chamber 1005a or the sample collection chamber 1005b.

Further, in response to the selection of one of the sample collection chambers via the selector device, the selector device connects a reservoir containing a processing fluid that is associated with that sample collection chamber to the chamber, e.g., in a manner discussed above.

Similar to the previous embodiments, one or more fluidic channels within the dermal patch may be evacuated to a pressure below the atmospheric pressure so as to facilitate the flow of various fluids (e.g., the physiological sample and/or the processing fluid) through those channels.

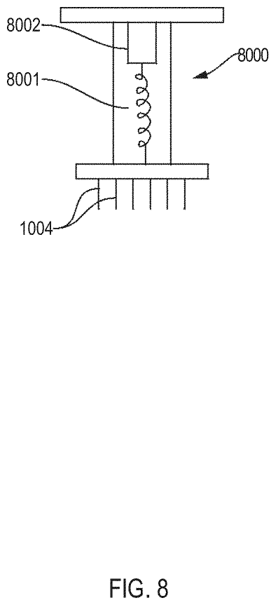

In some embodiments of any of the above dermal patches, the microneedles can be transitioned from a retracted position to a deployed position for puncturing the skin. By way of example, with reference to FIG. 8, in some such embodiments, an actuation mechanism 8000 can move the microneedles between the retracted and the deployed position. The actuation mechanism can be activated, for example, mechanically or electromechanically. For example, in this embodiment, a spring 8001 can be transitioned between a compressed and an extended position via rotation of a knob 8002. In particular the rotation of the nob 8002 can cause linear motion of a piston 8003 coupled to one end of the spring so as to move the spring between a compressed and an extended state so as to transition the microneedles 1004 between a retracted and a deployed state, respectively.

In some embodiments, a dermal patch according to the present teachings may not include any reservoirs and the processing reagent(s) needed for processing a collected sample may be stored within the sample collection chambers. By way of example, such a dermal patch may be similar to that shown in FIG. 1 but without the reservoirs and their associated fluidic channels. In some implementations of such a dermal patch, the processing fluid may be stored in a sample collection chamber in a lyophilized form, and can be reconstituted upon the introduction of a collected sample into the sample collection chambers. Further, in some embodiments, some of the sample collection chambers may contain the requisite sample processing reagent(s) while other sample collection chambers may be coupled to reservoirs for receiving the processing reagent(s).

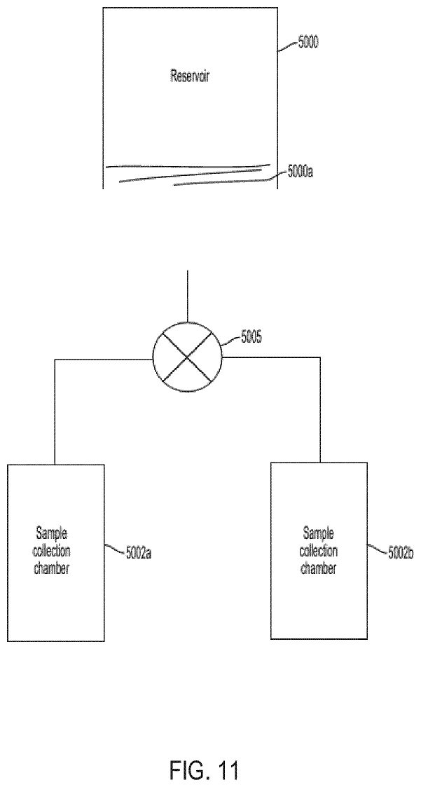

Further, in some embodiments, a dermal patch according to the present teachings may be configured such that two or more of the sample collection chambers share a reservoir. By way of example, FIG. 11 is a partial schematic view of such a dermal patch, which includes a single reservoir 5000 containing processing reagents 5000a, which is shared between two sample collection chambers 5002a/5002b. A switch 5005 can be activated to direct the processing reagent(s) stored in the reservoir into one or the other of the sample collection chambers.

A dermal patch according to the present teachings, such as the above dermal patches, may be employed to detect a variety of physiological target analytes in a sample drawn from a subject, e.g., a blood sample and/or an interstitial fluid sample, including a variety of biomarkers. Some examples of such target analytes include, without limitation, Cardiac troponin I protein (cTnI), Cardiac troponin T protein (cTnT), C-reactive protein (CRP), B-type natriuretic peptide (BNP), Myeloperoxidase, Creatine kinase MB, Myoglobin, Hemoglobin, HbA1C.

Further, in some embodiments, the dermal patch may be configured to detect one or more biomarkers for diagnosis of brain damage, such as traumatic brain injury (TBI). Some examples of such biomarkers include, without limitation, myelin basic protein (MBP), ubiquitin carboxyl-terminal hydrolase isoenzyme L1 (UCHL-1), neuron-specific enolase (NSE), glial fibrillary acidic protein (GFAP), and S100-B.

By way of example, the dermal patch may be configured to measure levels of the protein biomarkers UCHL-1 and GFAP, which are released from the brain into blood within 12 hours of head injury. The levels of these two proteins measured by a dermal patch according to the present teachings after a mild TBI can help identify those patients that may have intracranial lesions. In some such implementations of a dermal patch according to the present teachings, each sensing unit associated with a sample collection chamber can have at least two sensors one of which is configured for the detection of UCHL-1 and the other for the detection of GFAP. In addition or alternatively, the dermal patch may include one set of sample collection chambers dedicated to the detection UCHL-1 and another set of sample collection chambers that are dedicated to the detection of GFAP. The dermal patch may then be employed to obtain the levels of these proteins in blood samples drawn from a patient at different times, thereby facilitating the diagnosis of TBI.

In some embodiments, a dermal patch according to the present teachings, such as those discussed above, may include a controller that can be programmed to cause the collection of a sample (e.g., a blood sample) according to a predefined temporal schedule. By way of example, with reference to FIGS. 7A and 7B, in some implementations of the above dermal patch 7000, the on-board circuitry may include computer system 7001 that may be programmed to activate the selector device 7002 so as to allow a physiological sample, e.g., a blood sample or an interstitial fluid sample, to be drawn into the sample collection chambers 7004a and 7004b at different times for analysis.

The computer system 7001 may be implemented in any of hardware, software and/or firmware in a manner known in the art as informed by the present teachings 70017002

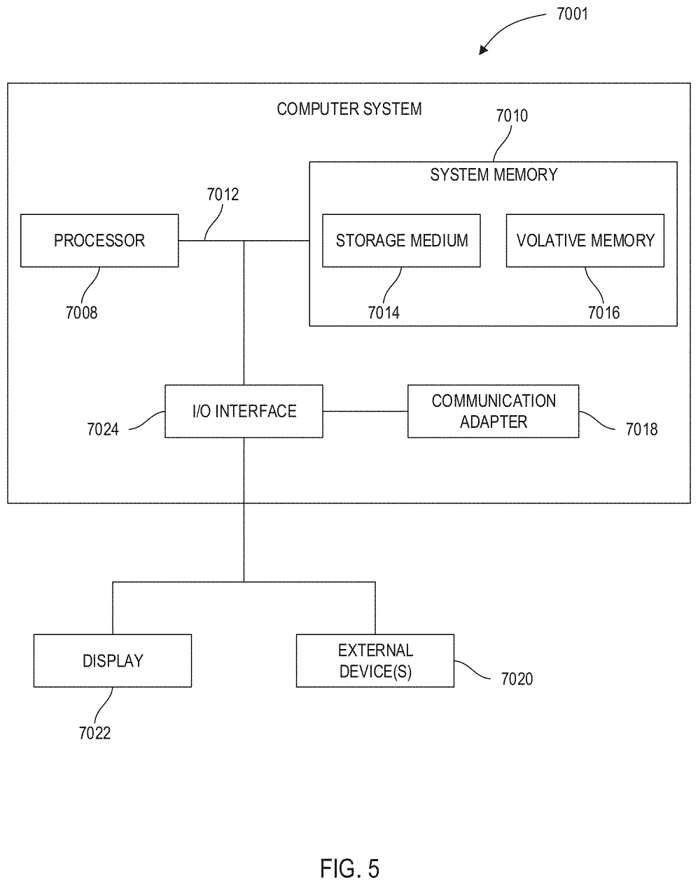

Referring now to FIG. 5, the computer system 7001 is shown in accordance with an exemplary embodiment. As used herein a computer system (or device) is any system/device capable of receiving, processing, and/or sending data. Computer systems include, but are not limited to, microprocessor-based systems, personal computers, servers, hand-held computing devices, tablets, smartphones, multiprocessor-based systems, mainframe computer systems, virtual reality (“VR”) headsets and the like.

As shown in FIG. 5, the computer system 7001 includes one or more processors or processing units 7008, a system memory 7010, and a bus 7012 that couples various components of the computer system 7001 including the system memory 7010 to the processor 7008.

The system memory 7010 includes a computer readable storage medium 7014 and volatile memory 7016 (e.g., Random Access Memory, cache, etc.). As used herein, a computer readable storage medium includes any media that is capable of storing computer readable program instructions and is accessible by a computer system. The computer readable storage medium 7014 includes non-volatile and non-transitory storage media (e.g., flash memory, read only memory (ROM), hard disk drives, etc.). Computer readable program instructions as described herein include program modules (e.g., routines, programs, objects, components, logic, data structures, etc.) that are executable by a processor. Furthermore, computer readable program instructions, when executed by a processor, can direct a computer system (e.g., the computer system 7001) to function in a particular manner such that a computer readable storage medium (e.g., the computer readable storage medium 7014) comprises an article of manufacture. Specifically, when the computer readable program instructions stored in the computer readable storage medium 7014 are executed by the processor 7008 they create means for activating the switch 7004 according to a predefined temporal schedule, e.g., for collecting a physiological sample may be stored in the computer readable storage medium 7014, e.g., at times separated from one another by one hour.

The bus 7012 may be one or more of any type of bus structure capable of transmitting data between components of the computer system 7001 (e.g., a memory bus, a memory controller, a peripheral bus, an accelerated graphics port, etc.).

The computer system 7001 may further include a communication adapter 7018 which allows the computer system 7001 to communicate with one or more other computer systems/devices via one or communication protocols (e.g., Wi-Fi, BTLE, etc.) and in some embodiments may allow the computer system 7001 to communicate with one or more other computer systems/devices over one or more networks (e.g., a local area network (LAN), a wide area network (WAN), a public network (the Internet), etc.).

In some embodiments, the computer system 7001 may be connected to one or more external devices 7020 and a display 7022. As used herein, an external device includes any device that allows a user to interact with a computer system (e.g., mouse, keyboard, touch screen, etc.). An external device 7020 and the display 7022 may be in communication with the processor 7008 and the system memory 7010 via an Input/Output (I/O) interface 7024.

The display 7022 may display a graphical user interface (GUI) that may include a plurality of selectable icons and/or editable fields. A user may use an external device 7020 (e.g., a mouse) to select one or more icons and/or edit one or more editable fields. Selecting an icon and/or editing a field may cause the processor 7008 to execute computer readable program instructions stored in the computer readable storage medium 7014. In one example, a user may use an external device 7020 to interact with the computer system 7001 and cause the processor 7008 to execute computer readable program instructions relating to at least a portion of steps of the methods disclosed herein.

While FIG. 7A depicts the dermal patch 7000 as including the computer system 7001, in some embodiments, the computer system 7000 may be omitted.

A dermal patch as disclosed herein would allow monitoring one or more biomarkers at different discrete times. Such monitoring of a biomarker level at different discrete times may be employed, for example, in the diagnosis of a disease condition and/or the progression of a disease condition, and/or the response of a patient to a therapeutic regimen. By way of example, when a subject is suspected to have suffered from a traumatic brain injury (e.g., concussion), the monitoring of a TBI-related biomarker at a plurality of discrete times may help with diagnosis of TBI and its temporal progression.

In another application, a dermal patch according to the present teachings can be used to assess the progression of organ damage. By way of example, the dermal patch may be used to monitor the level of troponin in a cardiac patient at a plurality of discrete times to assess the temporal progression of damage to the patient's heart muscle tissue.

In other embodiments, rather than employing a pre-programmed schedule of sample collection, the patch can allow, e.g., via the activation of the selector device by a user (e.g., a patient and/or a healthcare provider), to collect a plurality of physiological samples (e.g., blood samples) from the patient at different times based on the user's decision.

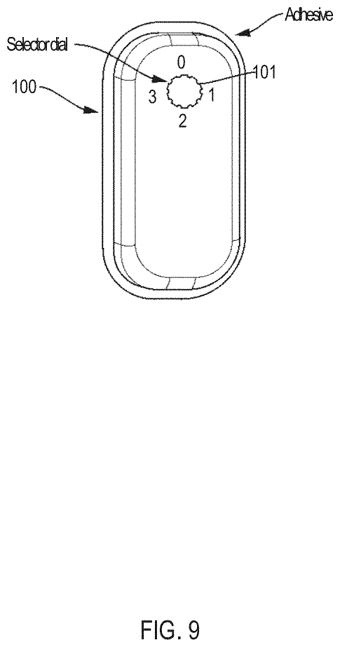

In many embodiments, the selector device of a dermal patch according to the present teachings may include a visual indicator that allows a user to select one of the sample collection chambers for receiving a physiological sample and/or a sample processing fluid. By way of example, as shown schematically in FIG. 9, a dermal patch 100 according to the present teachings includes a selector dial 101 having a visual indicator in the form of an arrow that can be aligned with each of a plurality of reference numerals, each associated with one of the sample collection chambers, to select the sample collection chamber associated with the selected reference numeral.

In some embodiments, a dermal patch according to the present teachings may include an indicator that shows which of the sample collection chambers have already been used for collecting the physiological sample. By way of example, such an indicator may be a light indicator that changes color, e.g., from green to red, to indicate that a sample collection chamber contains a sample.

Although in the above embodiments, the dermal patches are depicted to include two sample collection chambers, the present teachings are not restricted to dermal patches having only two sample collection chambers. For example, in other embodiments, four or more (e.g., up to 10), sample collection chambers may be employed.

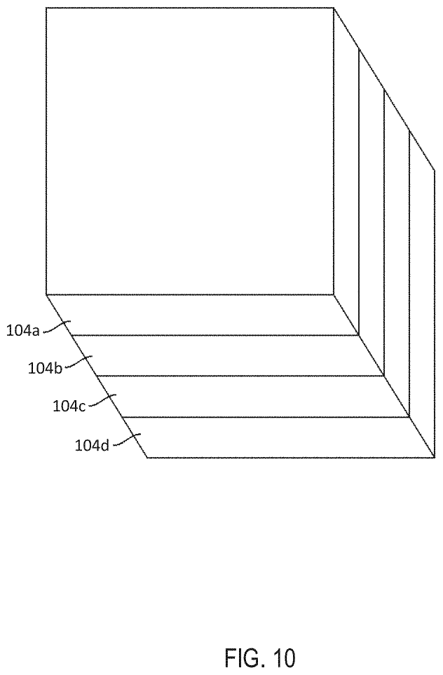

By way of illustration, with reference to FIGS. 9 and 10, the dermal patch 100 includes four sample collection chambers 104a, 104b, 104c, 104d, which are configured for collection of a physiological sample. In this embodiment, the sample collection chambers are in the form of parallelopipeds, though in other embodiments other shapes may be used. Further, while in this embodiment the sample collection chambers are stacked next to each other along the horizontal direction, i.e., with their long dimensions perpendicular to the top and bottom surfaces of the patch, in other embodiments, the sample collection chambers may be stacked along an orthogonal direction (herein also referred to as the “vertical direction”), i.e., with their long dimensions parallel to the two opposed surfaces of the patch, or may be stacked relative to one another in any other suitable configuration. Similar to the previous embodiments, each of the sample collection chambers may be associated with a respective reservoir in which a processing fluid may be stored.

In some embodiments, a dermal patch, such as those disclosed above, does not incorporate an electronic circuitry and/or a power supply. For example, in some such embodiments, a colorimetric sensor, such as an immunoassay sensor (e.g., a lateral flow immunosensor) with or without isothermal amplification of a target analyte, may be incorporated into the dermal patch to allow detecting (and optionally quantifying) a target analyte without a need to supply power to the detector. The output of such a sensor can be observed chromatographically and/or via “photo-visual” read.

Alternatively, a dermal patch having a sensor that requires power may be energized externally. For example, such a sensor may receive power via a mobile device, such as a wearable device. In other words, in some embodiments, the dermal patch together with a wearable device that can supply power to the dermal patch provide a modular system. By way of example, such transfer of power from an external source to the sensor can be achieved via an inductive coupling between the sensor and the external power source.

A dermal patch according to the present teachings can be attached to any suitable site of a subject's body. By way of example, the dermal patch may be attached to a subject's arm, e.g., a forearm or an upper arm. The attachment of the dermal patch to a subject's arm can be particularly convenient in cases when a wearable device may be employed for supplying power to the dermal patch, e.g., in a manner discussed above. In some such embodiments, the sensor can include a communication module that allows transmitting signals generated by the sensor to an external device, e.g., a wearable device being worn by the user, for analysis and presentation.

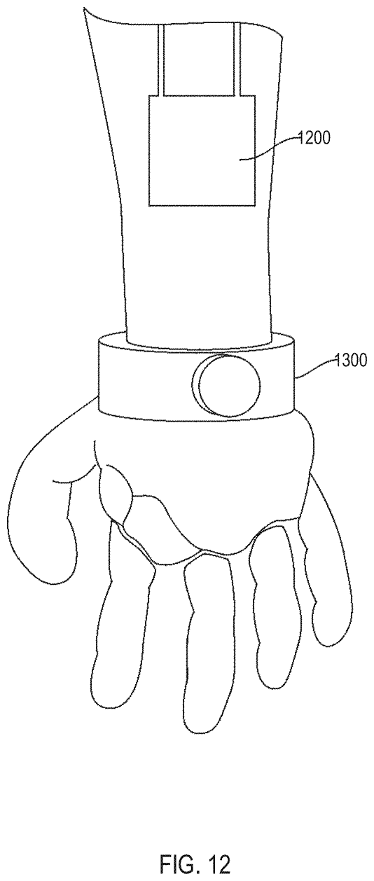

For example, as shown schematically in FIG. 12, in some embodiment, the circuitry and/or the sensors associated with a dermal patch 1200 according to the present teachings may be supplied with power via a wearable device 1300 worn by a subject. Although FIG. 12 shows the dermal patch and the wearable device being worn on the same arm, it may be more convenient for the subject to wear the dermal patch on one arm and the wearable device on the other arm so as to allow the wearable device to be brought into close proximity, and/or in contact, with the dermal patch for supplying power thereto. Alternatively, the dermal patch and the wearable device may be worn on the same arm in sufficient proximity to one another to allow transfer of power and/or data therebetween.

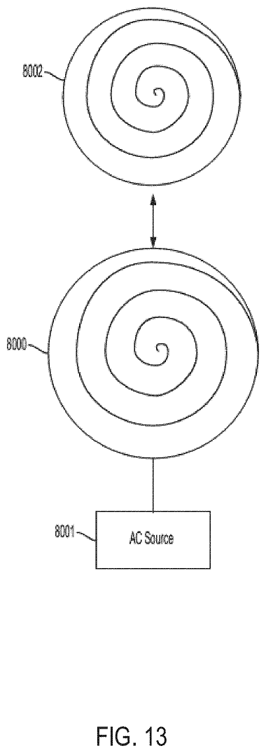

In some embodiments, an inductive coupling between the wearable device and the dermal patch may be employed for transferring power from the wearable device (or another external device) to the dermal patch. For example, as shown schematically in FIG. 13, the wearable device may include a coil (or a stack of coils) 8000 that may be energized via a power source 8001 (typically an AC power source) to generate an oscillating magnetic field, which can be inductively coupled to a respective coil 8002 incorporated in the dermal patch to generate a current in the coil 8002. Any other suitable means for transferring power from the wearable device to the dermal patch may also be employed. In some embodiments, the teachings disclosed in “Wireless Technologies for Implantable Device,” published on Aug. 16, 2020 online in PubMed (PMID: 32824365), which is incorporated herein by reference in its entirety, as informed by the present teachings, may be employed for communicating and/or supplying power to a dermal patch according to the present teachings.

Further, as noted above, in some embodiments, a dermal patch according to the present teachings may include communication circuitry, such as Bluetooth, for transmitting data (e.g., signals generated by the sensors) to an external device, such as the wearable device discussed above.

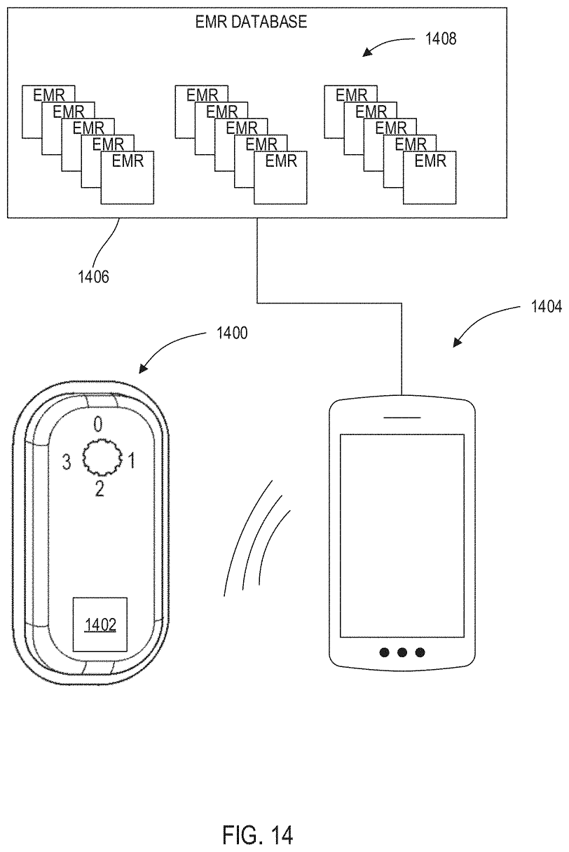

Referring now to FIG. 14 a dermal patch 1400 is shown in accordance with an exemplary embodiment and is similar to the dermal patches previously described herein and may include the dermal patch 7000. Like features previously described herein retain the same reference numbers and for the sake of brevity, description thereof will not be repeated.

In this embodiment, a quick response (“QR”) code 1402 is printed onto a top surface of the dermal patch 1400. In this embodiment, a user may install an application stored as computer readable program instructions on a computer system 1404 (i.e., a smartphone, tablet, etc.) and employ a camera of the computer system 1404 to take a photo of the QR code 1402 which is saved in a memory of the computer system 1404. Generally, the computer system 1404 includes same or similar components as the computer system 7001 (i.e., system memory, processor, display, etc.). In this embodiment, a processor of the computer system 1404 may execute the program instructions associated with the application to retrieve the photograph from the memory.

In some embodiments, the computer system 1404 may be in communication with an electronic medical record (“EMR”) database 1406 via a network connection. The EMR database 1406 includes a plurality of EMRs 1408 each associated with an individual subject. In these embodiments, the instructions associated with the application further cause the processor of the computer system 1404 to analyze the photograph to identify the QR code 1402 and associate the QR code 1402 with an EMR 1408 stored in the EMR database 1406. When a sensor 3000 of the derma patch 1400 includes a visible readout (e.g., a colorimetric sensor) and the readout is included in the photograph, the processor of the computer system 1404 may further analyze the received photo to evaluate the readout and automatically determine the presence of a target analyte and/or a level of a target analyte based on the readout as previously discussed herein.

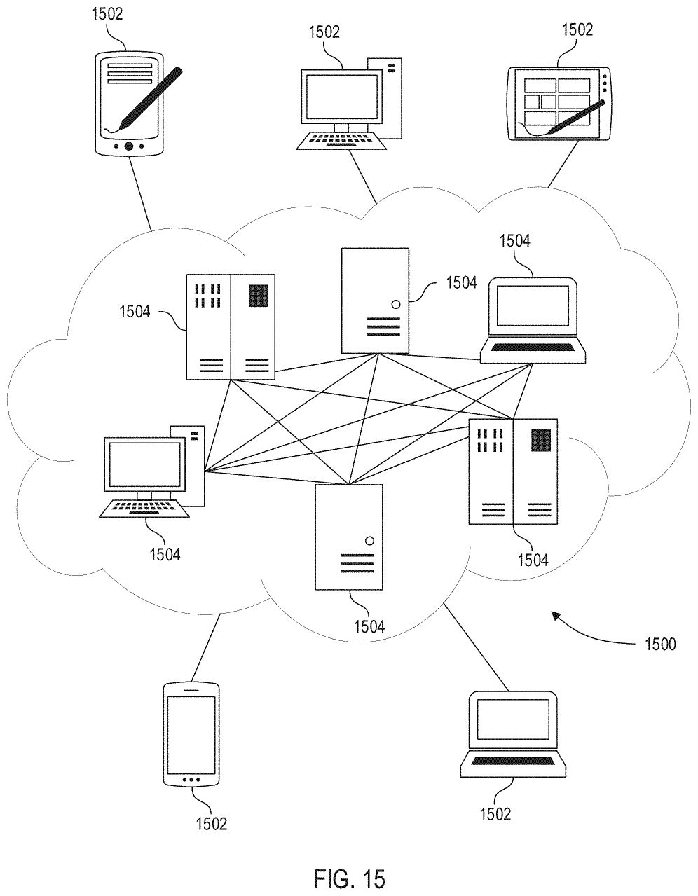

Referring now to FIG. 15, a cloud computing environment 1500 is depicted in accordance with an exemplary embodiment. The cloud computing environment 1500 is connected to one or more user computer systems 1502 and provides network access to shared computer resources (i.e., storage, memory, applications, virtual machines, etc.) to the one or more user computer systems 1502. As depicted in FIG. 15, the cloud computing environment 1500 includes one or more interconnected nodes 1504. Each node 1504 may be a computer system or device with local processing and storage capabilities. The nodes 1504 may be grouped and in communication with one another via one or more networks. This allows the cloud computing environment 1500 to offer software services to the one or more user computer systems 1502 and as such, a user computer system 1502 does not need to maintain resources locally.

In one embodiment, a node 1502 includes the computer system 7000 or the computer system 1404 and as such, includes the computer readable program instructions for carrying out various steps of the methods discussed herein. In these embodiments, a user of computer system 1502 that is connected to the cloud computing environment 1500 may cause a node 1504 to execute the computer readable program instructions to carry out various steps of the methods disclosed herein.

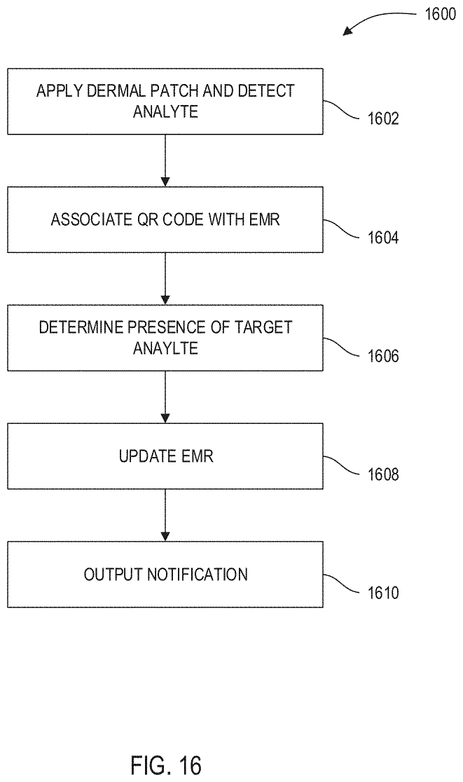

Referring now to FIG. 16, a method 1600 for automatically updating an EMR is shown in accordance with an exemplary embodiment. Steps 1604-1610 of the method 1600 may be stored as computer readable program instructions in a computer readable storage medium (e.g., memory of the computer system 1502 memory of a node 1504, etc.) a processor (e.g., a processor of the computer system 1404, a processor of a node 1504, etc.) executes the computer readable instructions for the steps 1604-1610 of the method 1600.

At 1602, the dermal patch 1400 is applied to the skin of a subject, and is activated to draw a physiological sample form the subject (e.g., a blood sample or a sample of interstitial fluid and the sensor 3000 detects an analyte as previously discussed herein).

At 1604, a user of the computer system 1404 scans the QR code 1402 with a camera of the computer system 1404 and a processor analyzes the QR code 1402 and associates the QR code 1402 with an EMR 1408 as previously discussed herein.

At 1606, the processor analyzes an image of the detector read out (e.g., bands in a lateral flow strip detector) to evaluate the readout of the sensor 3000 and automatically determine whether a target analyte is present in a physiological sample drawn from the subject, and optionally quantify the target analyte if the target analyte is detected in the sample as previously discussed herein.

At 1608, the processor automatically updates the associated EMR to include the determined presence of the target analyte and/or a level of the target analyte. In some embodiments, at 1608, the processor also updates the associated EMR to include the photograph of the QR code and the sensor 3000.

At 1610, the processor outputs a notification indicative of the determined presence of the target analyte and/or the determined level of the target analyte to a display in communication with the processor and/or outputs a notification indicative of the determined presence of the target analyte and/or the determined level of the target analyte to another device (e.g., a physician's smartphone).

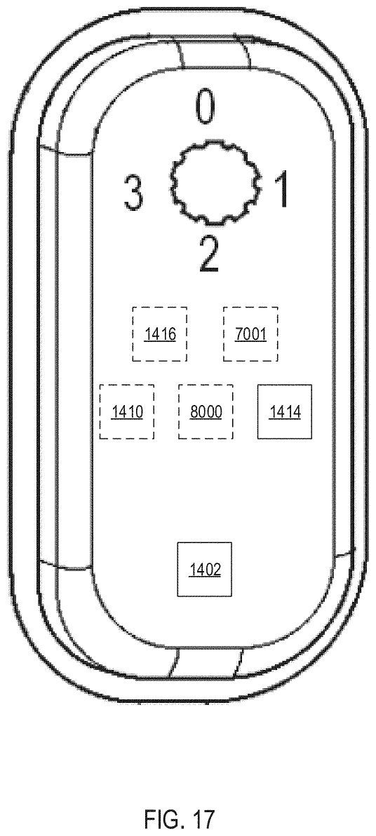

Referring now to FIG. 17, in some embodiments the dermal patch 1400 may further include an electromechanical actuator 1410 that is coupled to and in communication with the selector device 1010 and the actuation mechanism 8000. In this embodiment, the electromechanical actuator is configured to move the selector device 1010 as previously discussed herein and is further configured to cause the actuation mechanism 8000 to move the microneedles 1004 between the retracted and deployed state via rotation of the knob 8002 as previously discussed herein.

The electromechanical actuator 1410 is also connected to and in communication with the computer system 7001. As such the electromechanical actuator 1410 is connected to and in communication with the processor 7008. In some embodiments, the electromechanical actuator 1410 is wirelessly connected to the computer system 7001 and in other embodiments the connection between the electromechanical actuator 1410 and the computer system 7001 is a wired connection. The electromechanical actuator 1410 is configured to move the selector device 1010 and cause the actuation mechanism 8000 to move the microneedles 1004 to the deployed position in response to receiving a signal from the processor 7008.

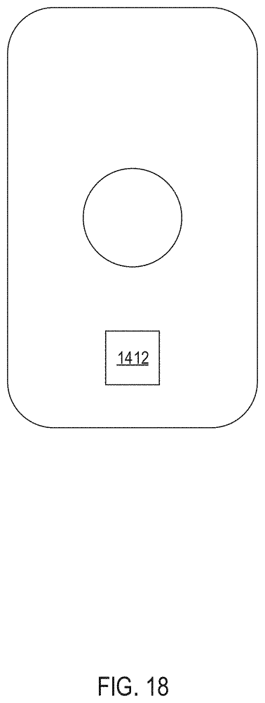

Referring now to FIG. 18, in some embodiments, the dermal patch 1400 may further include a skin sensor 1412 located on a bottom surface of the dermal patch 1400. The skin sensor 1412 is configured to determine when the dermal patch 1400 is adhered to skin of a subject. Stated another way, the skin sensor 1412 is configured to determine when the bottom surface of the dermal patch 1400 contacts skin of the subject. The skin sensor 1412 includes, but is not limited to, optical sensors, infrared sensors, light sensors, temperature sensors, pulse sensors, etc.

The skin sensor 1412 is connected to and in communication with the computer system 7001. As such, the skin sensor 1412 is connected to and in communication with the processor 7008. In some embodiments, the skin sensor 1412 is wirelessly connected to the computer system 7001 and in other embodiments, the connection between the skin sensor 1412 and the computer system 7001 is a wired connection. In response to determining the dermal patch 1400 is adhered to the skin of the subject, the skin sensor 1412 sends a signal to the processor 7008 indicating that the dermal patch 1400 is adhered to the subject.

In some embodiments, in response to receiving the signal indicating that the dermal patch 1400 is adhered to the subject, the processor 7008 sends a signal to the electromechanical actuator 1410 to deploy the needles. In response to receiving the signal to deploy the needles, the electromechanical actuator 1410 causes the actuation mechanism 8000 to move the microneedles 1004 to the deployed position via rotation of the knob 8002. Stated another way, in response to the skin sensor 1412 determining the dermal patch 1400 is adhered to a subject, the processor 7008 automatically causes the dermal patch 1400 to draw a physiological sample as previously discussed herein. In some embodiments, the processor 7008 causes the actuation mechanism 8000 to move the microneedles 1004 to the deployed position after a given amount of time has passed since the skin sensor 1412 determined the dermal patch 1400 was adhered to the subject (e.g., 5 seconds, 10 seconds, 15 seconds, etc.).

As depicted in FIG. 17, the dermal patch 1400 may include an actuation button 1414 and a locking mechanism 1416 (e.g., a pin that prevents mechanical movement of devices coupled to the locking mechanism when in a “locked” position and allows mechanical movement of devices coupled to the locking mechanism when in an “unlocked” position). The actuation button 1414 and the locking mechanism 1416 are connected to and in communication with the actuation mechanism 8000. The locking mechanism 1416 is mechanically coupled to the actuation mechanism 8000 and is moveable between a locked state and an unlocked stated. In the locked state, the locking mechanism 1416 prevents the actuation mechanism 8000 from moving the microneedles 1004 to the deployed position, whereas in the unlocked state, actuation mechanism 8000 is capable of moving the microneedles 1004 to the deployed position. The actuation button 1414 is configured to cause the actuation mechanism 8000 to move the microneedles 1004 to the deployed state (e.g., via rotation of the knob 8002) when pressed. As such, after being adhered to a subject, a user may draw a physiological sample from the subject by pushing the actuation button 1414 when the locking mechanism 1416.

In another embodiment, the locking mechanism 1416 is mechanically coupled to the actuation button 1414. In this embodiment, the locking mechanism 1416 does not allow the actuation button 1414 to be depressed when in the locked state. Stated another way, when the locking mechanism 1416 is in the locked state, the actuation button 1414 is not capable of causing the actuation mechanism 8000 to move the microneedles 1004 to the deployed position. When the locking mechanism 1416 is in the unlocked state, the actuation button 1414 may be depressed. Stated another way, a user of the dermal patch 1400 may press the actuation button 1414 to obtain the physiological sample when the locking mechanism 1416 is in the unlocked state.

The electromechanical actuator is coupled to and in communication with the locking mechanism 1416. In one embodiment, after receiving a signal indicating the dermal patch 1400 is adhered to skin of the subject from the skin sensor 1412, the processor 7008 sends a signal to move the locking mechanism 1416 to the unlocked state thereby allowing a user to draw the physiological sample by pushing the actuation button 1414.

As previously discussed herein, a user may employ a camera of the computer system 1404 to scan the QR code 1402. In some embodiments, before scanning the QR code 1402, the previously discussed installed application may require a user to verify their identity (e.g., by entering a password, scanning a fingerprint, etc.). For example, the installed application may require a user to enter a username and password that is associated with an EMR. In response to verifying the identity of the user, the application may unlock thereby allowing the user to scan the QR code 1402. Furthermore, after the application verifies the identity of the user and in response to associating the QR code 1402 with the correct EMR as previously discussed herein, the computer system 1404 may send a signal indicating that the identity of the user has been verified to the processor 7008.

In some embodiments, in response to receiving the signal indicating that the identity of the user has been verified, the processor 7008 sends a signal to the electromechanical actuator 1410 to deploy the microneedles 1004 as previously discussed herein. In the embodiment wherein the dermal patch 1400 includes the locking mechanism 1416, in response to receiving the signal indicating that the identity of the user has been verified, the processor 7008 sends a signal to the electromechanical actuator 1410 to place the locking mechanism 1416 in the unlocked state as previously discussed herein.

In some embodiments, before sending the signal to electromechanical actuator 1410 to deploy the microneedles 1004 or sending the electromechanical actuator 1410 to place the locking mechanism 1416 in the unlocked state, the processor 7008 may only send the signal in response to receiving both the signal indicating that the user identity has been verified and the signal indicating that the dermal patch 1400 has been adhered to skin of the subject as previously discussed herein.

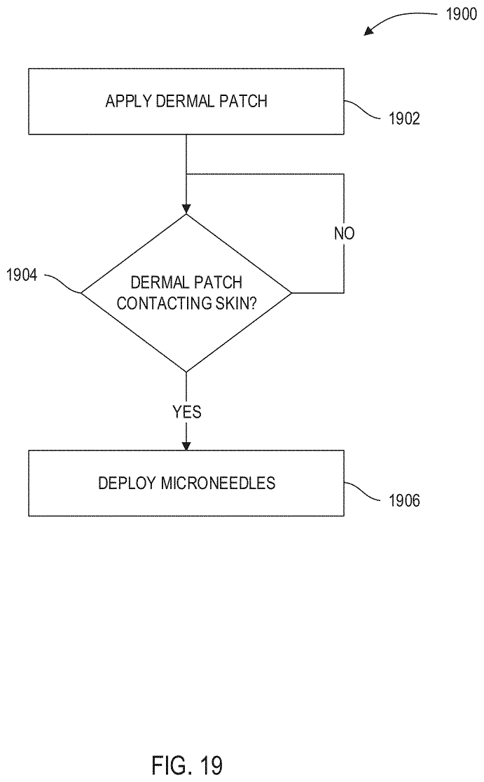

Referring now to FIG. 19, a method 1900 for drawing a physiological sample is shown in accordance with an exemplary embodiment. Steps 1904 and 1906 of the method 1900 may be stored as computer readable program instructions in a computer readable storage medium. A processor executes the computer readable program instructions for the steps 1904 and 1906 of method 1900.

At 1902, the dermal patch 1400 is applied to skin of a subject as previously discussed herein.

At 1904, the skin sensor 1412 determines if the dermal patch 1400 is applied to skin of the subject as previously discussed herein and in response to determining the dermal patch 1400 is adhered to skin of the subject, the skin sensor 1412 sends a signal to the processor 7008 indicating the dermal patch 1400 is adhered to skin.

At 1906, in response to receiving the signal indicating the dermal patch 1400 is adhered to the subject, the processor 7008 sends a signal to the electromechanical actuator 1410 to cause the actuation mechanism 8000 to deploy the microneedles 1004 to draw the physiological sample as previously discussed herein.

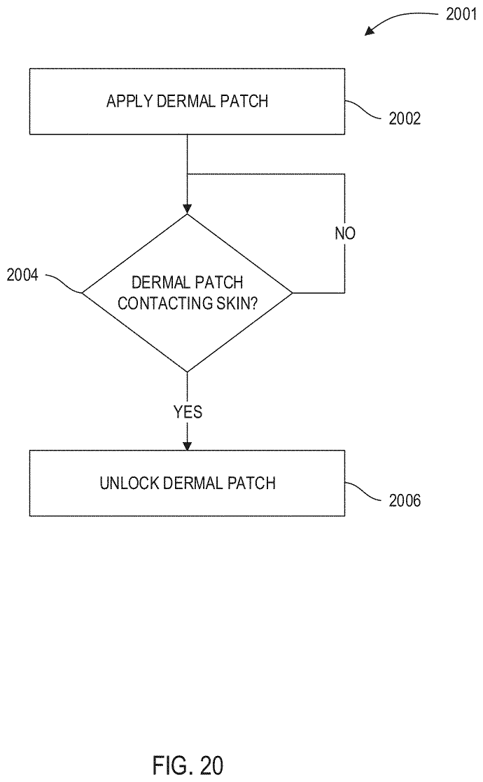

Referring now to FIG. 20, a method 2001 for placing the dermal patch 1400 in a state ready to draw a physiological sample is shown in accordance with an exemplary embodiment. Steps 2004 and 2006 of the method 2001 may be stored as computer readable program instructions in a computer readable storage medium. A processor executes the computer readable program instructions for the steps 2004 and 2006 of method 2001.

At 2002, the dermal patch 1400 is applied to skin of a subject as previously discussed herein.

At 2004, the skin sensor 1412 determines if the dermal patch 1400 is applied to skin of the subject as previously discussed herein and in response to determining the dermal patch 1400 is adhered to skin of the subject, the skin sensor 1412 sends a signal indicating the dermal patch 1400 is adhered to the processor 7008.

At 2006, in response to receiving the signal indicating the dermal patch 1400 is adhered to the subject, the processor 7008 sends a signal to the electromechanical actuator 1410 to place the locking mechanism 1416 in an unlocked position thereby allowing a user to draw a physiological sample as previously discussed herein.

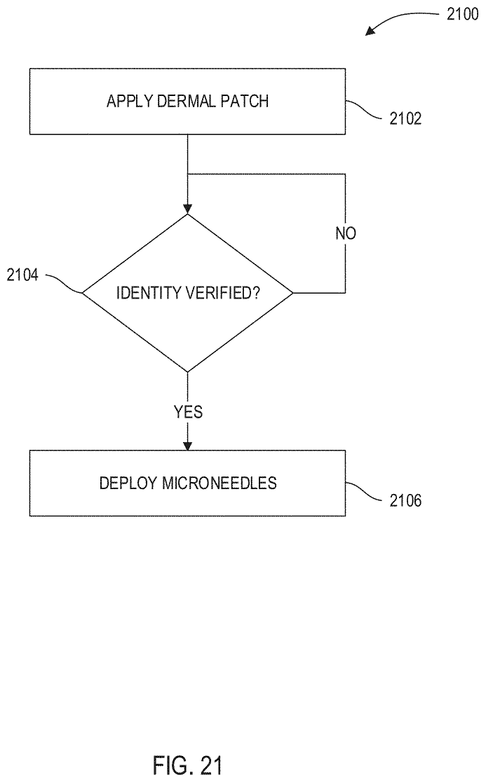

Referring now to FIG. 21, another method 2100 for drawing a physiological sample is shown in accordance with an exemplary embodiment. Steps 2104 and 2106 of the method 2100 may be stored as computer readable program instructions in a computer readable storage medium. A processor executes the computer readable program instructions for the steps 2104 and 2106 of method 2100.

At 2102, the dermal patch 1400 is applied to the skin of a subject as previously discussed herein.

At 2104, a user scans the QR code 1402 and the computer system 1404 verifies the identity of the user as previously discussed herein. In response to verifying the identity of the user, the computer system 1404 sends a signal indicating that the identity of the user has been verified to the processor 7008 as previously discussed herein.

At 2106, in response to receiving the signal indicating that the identity of the user has been verified, the processor 7008 sends a signal to the electromechanical actuator 1410 to cause the actuation mechanism 8000 to deploy the microneedles 1004 to draw the physiological sample as previously discussed herein.

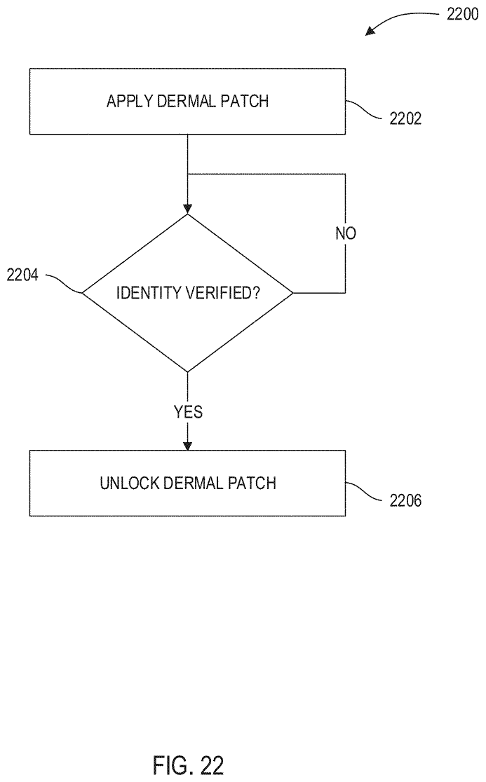

Referring now to FIG. 22, another method 2200 for placing the dermal patch 1400 in a state ready to draw a physiological sample is shown in accordance with an exemplary embodiment. Steps 2204 and 2206 of the method 2200 may be stored as computer readable program instructions in a computer readable storage medium. A processor executes the computer readable program instructions for the steps 2204 and 2206 of method 2200.

At 2202, the dermal patch 1400 is applied to the skin of a subject as previously discussed herein.

At 2204, a user scans the QR code 1402 and the computer system 1404 verifies the identity of the user as previously discussed herein. In response to verifying the identity of the user, the computer system 1404 sends a signal indicating that the identity of the user has been verified to the processor 7008 as previously discussed herein.

At 2206, in response to receiving the signal indicating that the identity of the user has been verified, the processor 7008 sends a signal to the electromechanical actuator 1410 to place the locking mechanism 1416 in an unlocked position thereby allowing a user to draw a physiological sample as previously discussed herein.

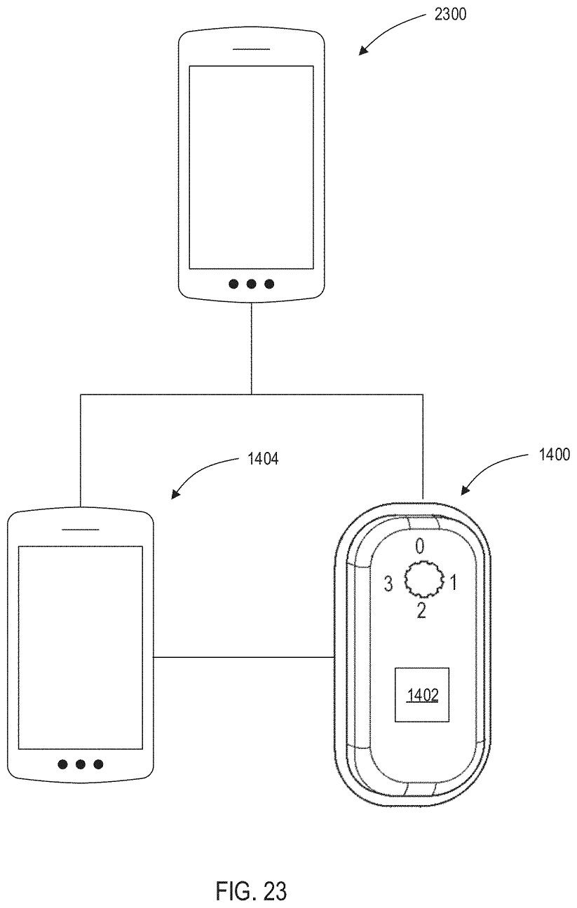

Referring now to FIG. 23, a medical professional's computer system 2300 is depicted in accordance with an exemplary embodiment. While FIG. 23 depicts the medical professional's computer system 2300 as a smartphone, in other embodiments the medical professional's computer system 2300 may be another type of computer system (e.g., a tablet, laptop, etc.). As depicted in FIG. 23, the medical professional's computer system 2300 may be connected to and in communication with one of or both of the computer system 1404 and the computer system 7001 (e.g., when the medical professional's computer system 2300, the computer system 1404, and/or the computer system 7001 are connected to a same network).

As previously discussed herein, the processor 7008 may receive a signal indicating that the dermal patch 1400 is adhered to the subject's skin from the skin sensor 1412 or a signal indicating that the identity of the user has been verified. In response to receiving one or both of these signals, the processor 7008 may send a signal indicating that the dermal patch 1400 is ready for operation to a processor of the medical professional's computer system 2300. In some embodiments, after verifying the identity of the user as previously discussed herein, a processor of the computer system 1404 sends a signal indicating that the dermal patch 1400 is ready for operation to the medical professional's computer system 2300.

In response to receiving the signal indicating that the dermal patch 1400 is ready for operation, the processor of the medical professional's computer system 2300 causes a display of the medical professional's computer system 2300 to display a notification indicating the dermal 1400 is ready for operation and displays a GUI with an actuatable icon that when selected by the medical professional sends a signal to deploy the microneedles 1004 or in the embodiment wherein the dermal patch 1400 includes the locking mechanism 1416 sends a signal to unlock the locking mechanism 1416 to the electromechanical actuator 1410 as previously discussed herein.

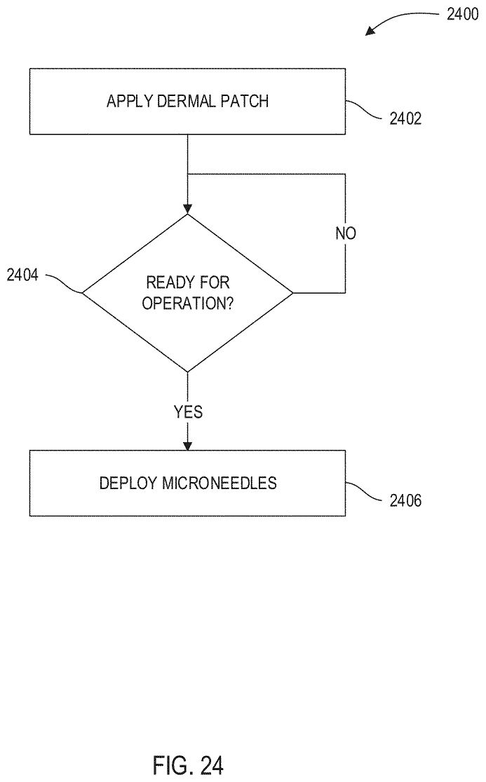

Referring now to FIG. 24, another method 2400 for drawing a physiological sample is shown in accordance with an exemplary embodiment. Steps 2404 and 2406 of the method 2400 may be stored as computer readable program instructions in a computer readable storage medium. A processor executes the computer readable program instructions for the steps 2404 and 2406 of method 2400.

At 2402, the dermal patch 1400 is applied to the skin of a subject as previously discussed herein.

At 2404, the processor 7008 sends a signal indicating the dermal patch 1400 is ready for operation to a medical professional's computer system 2300 in response to verifying an identity of a user and/or in response to determining the dermal patch 1400 is adhered to skin of a subject as previously discussed herein. Furthermore, at 2404, in response to a medical professional selecting an icon displayed in a GUI of a display of the medical professional's computer system 2300, the medical professional's computer system 2300 sends a signal to deploy the microneedles 1004 to the processor 7008 as previously discussed herein.

At 2406, in response to receiving the signal to deploy the microneedles 1004 from the medical professional's computer system 2300 the processor 7008 sends a signal to the electromechanical actuator 1410 to cause the actuation mechanism 8000 to deploy the microneedles 1004 to draw the physiological sample as previously discussed herein.

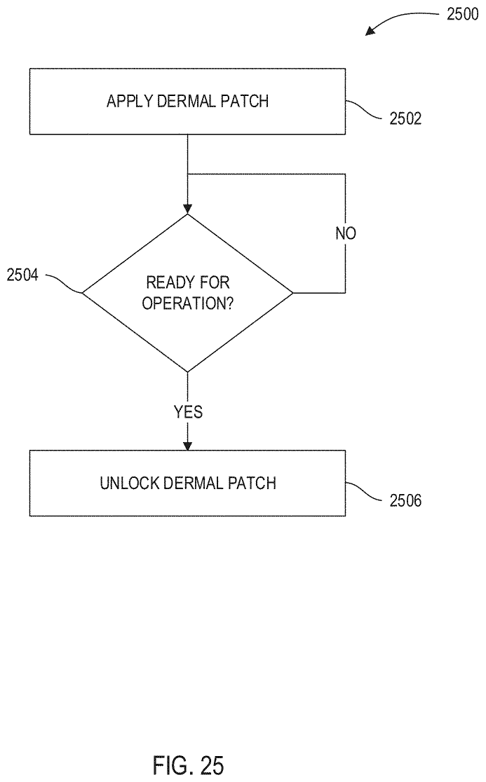

Referring now to FIG. 25, another method 2500 for placing the dermal patch 1400 in a state ready to draw a physiological sample is shown in accordance with an exemplary embodiment. Steps 2504 and 2506 of the method 2500 may be stored as computer readable program instructions in a computer readable storage medium. A processor executes the computer readable program instructions for the steps 2504 and 2506 of method 2500.

At 2502, the dermal patch 1400 is applied to the skin of a subject as previously discussed herein.

At 2504, the processor 7008 sends a signal indicating the dermal patch 1400 is ready for operation to a medical professional's computer system 2300 in response to verifying an identity of a user and/or in response to determining the dermal patch 1400 is adhered to skin of a subject as previously discussed herein. Furthermore, at 2504, in response to a medical professional selecting an icon displayed in a GUI of a display of the medical professional's computer system 2300, the medical professional's computer system 2300 sends a signal to unlock the locking mechanism 1416 as previously discussed herein.

At 2506, in response to receiving the signal to unlock the locking mechanism 1416, the processor 7008 sends a signal to the electromechanical actuator 1410 to place the locking mechanism 1416 in an unlocked position thereby allowing a user to draw a physiological sample as previously discussed herein.

While the methods 1900, 2001, 2100, 2200, 2400, and 2500 include the processor 7008 causing the electromechanically actuator 1410 to deploy the microneedles 1004 or include the processor 7008 causing the electrotechnical actuator 1410 unlocking the dermal patch 1400 in response to receiving one of a signal indicating the dermal patch 1400 is adhered to the subject or a signal indicating that the identity of the user has been verified or in response to receiving a signal from the medical professional's computer system 2300, in other embodiments, the processor 7008 sends the signal deploy the microneedles 1004 or unlock the dermal patch 1400 in response to receiving more than one of the previously recited signals.

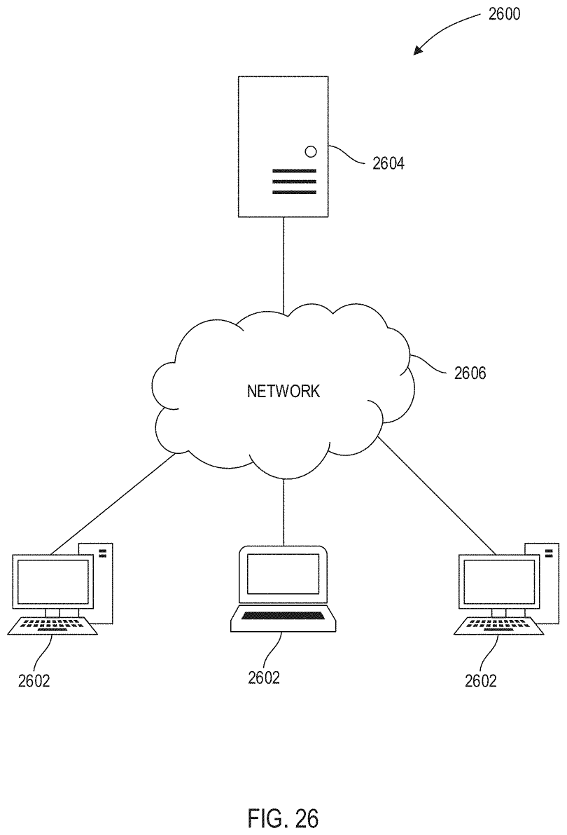

Referring now to FIG. 26, a metaverse network 2600 is shown in accordance with an exemplary embodiment. The metaverse network 2600 includes a plurality of user computer systems 2602, a metaverse server 2604, and a network 2606. In some embodiments, the computer systems 2602 may include one or more of the computer system 7001, the computer system 1404 and the medical professional's computer system 2300. While FIG. 26 depicts the metaverse network 2600 as including three user computer systems 2602 and one metaverse sever 2604, in other embodiments the metaverse network 2600 may include more or less user computer systems 2602 (e.g. 2, 5, 7, etc.) and more than one metaverse server 2604 (e.g., 2, 3, 6, etc.). The user computer systems 2602 are connected to and interface with the metaverse server 2604 via a network (e.g., a local area network (LAN), a wide area network (WAN), a public network (the Internet), etc.).

The metaverse server 2604 hosts a virtual reality environment and/or an augmented reality environment (hereinafter “a metaverse”) with which the users of a computer system 2602 may interact. In one embodiment, a specified area of the metaverse is simulated by a single server instance and the metaverse server 2604 may include a plurality of instances. The metaverse server 1604 may also include a plurality of physics servers configured to simulate and manage interactions, collisions, etc. between characters and objects within the metaverse. The metaverse server 2604 may further include a plurality of storage servers configured to store data relating to characters, media, objects, related computer readable program instructions, etc. for use in the metaverse.

The network 2606 may employ traditional internet protocols to allow communication between user computer systems 2602 and the metaverse server 2604. In some embodiments, the user computer systems 2602 may be directly connected to the metaverse server 2604.

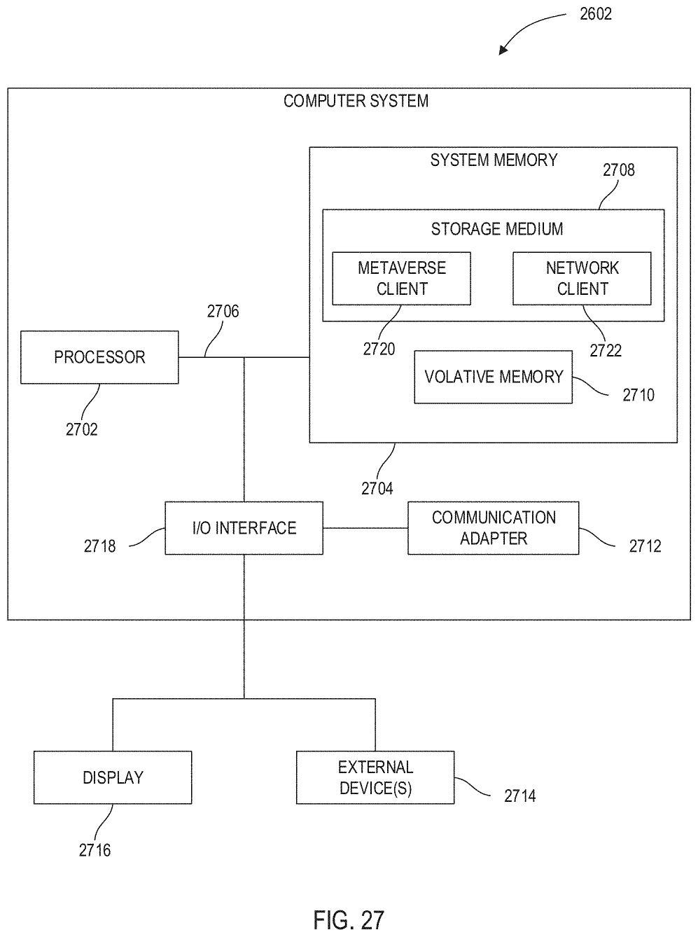

Referring now to FIG. 27 a user computer system 2602 is shown in accordance with an exemplary embodiment. Generally, the user computer system 2602 includes the same or similar components that operate in a same or similar manner as the components of the computer system 7001 (i.e., a processor 2702, system memory 2704, a bus 2706, a computer readable storage medium 2708, volatile memory 2710, a communication adapter 2712, one or more external devices 2714, a display 2716, and an I/O interface 2718). For the sake of brevity, these components are shown, but are not discussed in further detail herein.

The computer system 2602 also includes a metaverse client 2720 and a network client 2722. The metaverse client 2720 and the network client 2722 include computer readable program instructions that may be executed by a processor 2702 of the user computer system 2602. While FIG. 27 depicts the computer readable storage medium 2708 as including the metaverse client 2720 and the network client 2722, in other embodiments the metaverse client 2720 and the network client 2722 may be stored in a different location that is accessible to the processor 2702 (e.g., in a storage element of the cloud computing environment 1500).

When executed, the metaverse client 2720 allows a user of a computer system 2602 to connect to the metaverse server 2604 via the network 2606 thereby allowing a user of the user computer system 2602 to interact with the metaverse provided by the metaverse server 2604. The metaverse client 2720 further allows a user of a user computer system 2602 to interact with other users of other computer systems 2602 that are also connected to the metaverse server 2604.

The network client 2722, when executed by the processor 2702, facilities connection between the user computer system 2602 and the metaverse server 2604 (e.g., by verifying credentials provided by the user). For example, when executed and a user of a computer system 2602 requests to log onto the metaverse server 2604, the network client 2722 maintains a stable connection between the user computer system 2602 and the metaverse server 2604 and handles commands input by a user of a computer system 2602 and handles communications from the metaverse server 2604.

When a user of the user computer system 2602 is logged into the metaverse server 2604, the display 2716 conveys a visual representation of a metaverse provided by the metaverse server 2604. In some embodiments wherein a computer system 2602 is a VR headset and the VR headset includes the display 2716, the metaverse server 2604 provides a three-dimensional (“3D”) environment to the VR headset thereby creating a lifelike environment for the user.

In one embodiment, wherein the computer systems 7001 and 1404 are user computer systems 2602 (and therefore include the metaverse client 2720 and the network client 2722), a user of the dermal patch may log into the metaverse server 2604 by verifying their identity as previously discussed herein. In response to verifying the identity of a user, the computer system 7001 sends a signal indicating the user identity has been verified to the metaverse server 2604 and thereby logging the computer systems 7001 and 1404 into the metaverse.

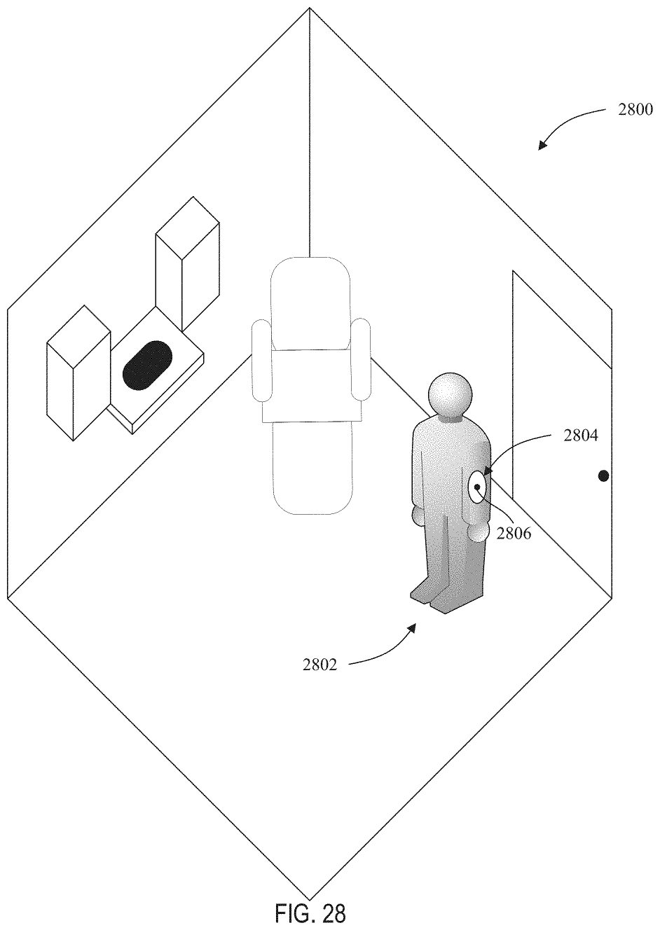

Referring now to FIG. 28, when a user computer system 2602 logs into the metaverse server 2604, the metaverse server 2604 generates a virtual reality environment (or a “metaverse”) 2800 and may generate a subject avatar 2802 corresponding to a user of the dermal patch 1400. In some embodiments, the metaverse 2800 looks like a physician's examination room (e.g., including chairs, examination table, sink, etc.) and may be based on user inputs to create a personalized metaverse 2800. After the subject avatar 2802 is generated, the metaverse server 2604 populates the subject avatar 2802 into the metaverse 2800. In some embodiments, the metaverse server 2604 generates a generic subject avatar 2802 that corresponds to the user and in other embodiments, the subject avatar 2802 has been previously generated by the metaverse based on user inputs. When the subject avatar 2802 is based on user inputs, the avatar may look similar to a subject using the dermal patch 1400.

Furthermore when the computer system 7001 and/or the computer system 1404 is a user computer system 2602 and is logged into the metaverse server 2604, in response to the skin sensor 1412 determining the dermal patch 1400 is contacting skin of the subject and sends a signal to the computer system 7001 or the computer system 1404 indicating the dermal patch 1400 is adhered to the subject as previously discussed herein, the computer system 7001 or the computer system 1404 may send a corresponding signal to the metaverse server 2604. In response to receiving the signal indicating the dermal patch 1400 is adhered to skin of the subject, the metaverse server 2604 generates a virtual dermal patch 2804 on the subject avatar 2802. While the virtual dermal patch 2804 is depicted on an arm of the subject avatar 2802, in other embodiments, the virtual dermal patch 2804 may be depicted as attached to different parts of the subject avatar 2802 (e.g., on a leg of the subject avatar).

The virtual dermal patch 2804 includes an actuatable button 2806. When a user within the metaverse selects the actuatable button 2806, the metaverse server 2604 sends a signal to the processor 7008 of the dermal patch 1408 to deploy the microneedles 1004 or unlock the dermal patch 1400 as previously discussed herein. In response to receiving the signal from the metaverse server 2604, the processor 7008 causes the electromechanical actuator to deploy the microneedles 1004 or unlock the dermal patch 1400 as previously discussed herein. Stated another way, a user in the metaverse 2800 may deploy the microneedles 1004 or unlock the dermal patch 1400 by pushing a button 2806 of a virtual dermal patch 2804. In some embodiments, the actuatable button 2806 may only be actuated by a user of a computer system 2602 with specific login credentials (i.e., a medical professional).

In some embodiments, wherein a user computer system 2602 includes a VR headset that is connected to the metaverse server 2604, a user may view the metaverse 2800 via a display of the VR headset. Furthermore, when the metaverse 2800 includes the avatar 2802 with the virtual dermal patch 2804, the VR headset may track the hands of the user in the VR headset to determine when the user “pushes” (and therefore selects) the actuatable button 2806. In response to determining the user pushed the actuatable button 2806, the VR headset (the user computer system 2602) sends a signal to the metaverse server 2604 indicating a user has selected the actuatable button 2806. In response to receiving this signal, the metaverse server 2604 causes the dermal patch 1400 to deploy the microneedles 1004 or unlock.

In some embodiments, wherein a medical professional logs into the metaverse server 2604 via their login credentials, the metaverse server may populate a corresponding avatar (e.g., a medical professional avatar) into the metaverse 2800. In these embodiments, when the medical professional selects the actuatable button 2806 the metaverse server depicts the medical professional's avatar as interacting with the virtual dermal patch 2804.

Those having ordinary skill in the art will appreciate that various changes may be made to the above embodiments without departing from the scope of the present teachings.

What is claimed is:

1. A dermal patch, comprising:
at least a pair of sensing units each configured for detecting at least one analyte in a physiological sample,
at least one microneedle configured for puncturing the skin to allow collection of the physiological sample, and
a selector device for selecting any one of said sensing units for receiving at least a portion of said collected physiological sample for analysis thereof.

2. The dermal patch of claim 1, wherein said selector device comprises at least one visual indicator for allowing a user to select one of said sensing units.

3. The dermal patch of claim 2, wherein said visual indicator comprises a selector dial.

4. The dermal patch of claim 1, further comprising a plurality of reservoirs for storing one or more reagents for processing the physiological sample to generate a processed sample, wherein each of said reservoirs is associated with one of said sensing units.

5. The dermal patch of claim 4, wherein said one or more processing reagents are suitable for isothermal amplification of said analyte.

6. The dermal patch of claim 5, wherein each of the sensing units comprises a sample collection chamber for receiving at least a portion of said physiological sample in response to selection of said sensing unit by said selector device.

7. The dermal patch of claim 6, wherein said sample collection chamber has a volume equal to or less than about 2 milliliters.

8. The dermal patch of claim 6, wherein said sample collection chamber has a volume equal to or less than about 1 milliliter.

9. The dermal patch of claim 6, wherein said sample collection chamber has a volume in a range of about 10 microliters to about 1 milliliter.

10. The dermal patch of claim 6, further comprising at least one fluid channel having an inlet configured to receive the physiological sample through the punctured skin and an outlet through which the received sample can be introduced into a sample collection chamber of a selected one of said sensing units.

11. The dermal patch of claim 10, further comprising a switch for selectively coupling said outlet to one of said sample collection chambers.

12. The dermal patch of claim 11, further comprising a controller in communication with the switch for activating the switch in accordance with a predefined temporal schedule to collect multiple physiological samples at different times.

13. The dermal patch of claim 12, wherein said switch comprises a plurality of internal channels, wherein in one position of the switch one of said internal channels directs the received physiological sample into one of the sample collection chambers and in another position of the switch another one of said internal channels directs the received physiological sample into another one of the sample collection chambers.

14. The dermal patch of claim 12, further comprising a fluid transfer channel coupled to the selector device for establishing a fluid path between a selected sensing unit and a reservoir associated therewith.

15. The dermal patch of claim 14, wherein each of said reservoir and a respective sample collection chamber are positioned relative to one another such that gravity can facilitate transfer of said one or more processing reagents from the reservoir to the sample collection chamber.

16. The dermal patch of claim 11, wherein said switch comprises an electromechanical switch.

17. The dermal patch of claim 11, wherein said switch comprises a mechanical switch.

18. The dermal patch of claim 6, further comprising one or more magnetic beads stored in at least one of said sample collection chambers, wherein said magnetic beads can be activated via an external magnet for cause mixing of the physiological sample and the one or more processing reagents introduced into said at least one sample collection chamber.

19. The dermal patch of claim 6, wherein each of said sensing units further comprises a sensor in fluid communication with the sample collection chamber thereof for receiving at least a portion of said processed sample and to generate one or more detection signals in response to detection of said analyte.

20. The dermal patch of claim 19, wherein each of the sample collection chambers comprises an opening for providing fluid communication between the sample collection chamber and a respective sensor.

21. The dermal patch of claim 6, wherein each of said sample collection chambers is formed at least partially of a flexible material such that a volume thereof changes in response to introduction of any of the physiological sample and said one or more processing reagents therein.

22. The dermal patch of claim 19, further comprising circuitry in communication with said sensors for receiving said one or more detection signals and processing said signals to quantify a concentration of detected analyte in the sample.

23. The dermal patch of claim 19, wherein at least one of said sensors comprises a graphene-based sensor.

24. The dermal patch of claim 19, wherein at least one of said sensors comprises an electrochemical sensor.

25. The dermal patch of claim 19, wherein at least one of said sensors comprises a colorimetric sensor.

26. The dermal patch of claim 25, wherein said colorimetric sensor employs an immunoassay for detection of said analyte.

27. The dermal patch of claim 25, wherein said dermal patch comprises a transparent window to allow visualization of said colorimetric sensor.

28. The dermal patch of claim 22, wherein said circuitry comprises at least one memory module for storing said detection signals.

29. The dermal patch of claim 28, wherein said circuitry is configured to process the stored detection signals generated by different sensing units to determine a variation, if any, of said analyte level at discrete time intervals associated with activation of said sensing units.

30. The dermal patch of claim 22, wherein said circuitry comprises a communication module for communicating with an electronic device.

31. The dermal patch of claim 30, wherein said communication module is configured to provide wireless communication between the patch and the electronic device.

32. The dermal patch of claim 31, wherein said communication module employs a wireless communication protocol selected from any of Bluetooth, Wi-Fi, and BTLE protocol for establishing a communication link between said patch and said electronic device.

33. The dermal patch of claim 30, wherein said electronic device is a mobile device.

34. The dermal patch of claim 1, wherein said physiological sample comprises any of blood and interstitial fluid.

35. The dermal patch of claim 1, wherein said analyte comprises a biomarker.

36. The dermal patch of claim 35, wherein said biomarker is indicative of a diseased condition.

37. The dermal patch of claim 35, wherein said analyte comprises a biomarker indicative of organ damage.

38. The dermal patch of claim 35, wherein said analyte comprises at least one biomarker indicative of traumatic brain injury.

39. The dermal patch of claim 35, wherein said at least one biomarker comprises any of myelin basic protein (MBP), ubiquitin carboxyl-terminal hydrolase isoenzyme L1 (UCHL-1), neuron-specific enolase (NSE), glial fibrillary acidic protein (GFAP), and S100-B.

40. The dermal patch of claim 35, wherein said biomarker comprises any of troponin, BNP, and HbA1C.

41. The dermal patch of claim 1, wherein said selector device comprises a mechanical selection element.

42. The dermal patch of claim 1, wherein said selector device comprises an electromechanical selection element.

43. The dermal patch of claim 1, wherein said selector device comprises an electromagnetic selection element.

44. The dermal patch of claim 1, wherein said sensing units are configured to detect the same analyte in the sample.

45. The dermal patch of claim 1, wherein said sensing units are configured to detect different analytes in the sample.

46. The dermal patch of claim 1, wherein said at least a pair of sensing units comprises more than three sensing units.

47. The dermal patch of claim 1, wherein said at least one microneedle comprises a plurality of microneedles.

48. The dermal patch of claim 1, wherein said at least one microneedle is movable between a retracted position and a deployed position, wherein said at least one microneedle is capable of puncturing the skin in said deployed position.

49. The dermal patch of claim 48, further comprising an actuation mechanism operably coupled to the selector device for moving said at least one microneedle from said retracted position to said deployed position upon selection of any one of said sensing units via said selector device.

50. The dermal patch of claim 1, wherein said at least one microneedle comprises a plurality of microneedles.

51. The dermal patch of claim 1, wherein said microneedles has a length in a range of about 100 microns to about 1500 microns.

52. The dermal patch of claim 1, further comprising a mechanism for generating a negative pressure within said fluid channel to facilitate introduction of said physiological sample into said fluid channel.

53. The dermal patch of claim 1, wherein said mechanism comprises a pump.

54. The dermal patch of claim 1, further comprising an adhesive layer for attaching the dermal patch of the skin surface.

55. A dermal patch, comprising:
at least two sample collection chambers, each configured for receiving a physiological sample collected from a subject,
at least one reservoir for storing one or more processing reagents for processing the physiological sample so as to provide a processed sample
at least two detection units each operably coupled to one of said sample collection chambers for detecting a target analyte, when present in the collected sample.

56. The dermal patch of claim 55, further comprising a selector device for selecting any of said sample collection chambers for receiving the physiological sample.

57. The dermal patch of claim 55, further comprising one or more microneedles configured for puncturing the subject's skin.

58. A dermal patch comprising:
at least a pair of sensing units each configured for detecting at least one analyte in a physiological sample,
at least one microneedle configured for puncturing the skin to allow collection of the physiological sample, and
a quick response code associated with an electronic medical record.

59. A dermal patch comprising
at least a pair of sensing units each configured for detecting at least one analyte in a physiological sample,
at least one microneedle configured for puncturing the skin to allow collection of the physiological sample, and
computer system configured to connect to a metaverse.
